(19) United States
(12) Patent Application Publication
(10) Pub. No.: US 2024/0076714 A1
(43) Pub. Date: Mar. 7, 2024

(54) MICROFLUIDIC DEVICES AND RAPID PROCESSING THEREOF

(71) Applicant: HueDX, INC., Philadelphia, PA (US)

(72) Inventors: Reshma Rajan, Philadelphia, PA (US); Divykumar Patel, Philadelphia, PA (US); Nisarg Dave, Philadelphia, PA (US); Achal Shah, Philadelphia, PA (US); Akshay Gutha Ravichandran, Philadelphia, PA (US); Brittany Auyoung, Philadelphia, PA (US); Manjeet Dhindsa, Philadelphia, PA (US); Nidhi Menon, Philadelphia, PA (US)

(21) Appl. No.: 18/261,571

(22) PCT Filed: Jan. 20, 2022
(86) PCT No.: PCT/US22/13111

§ 371 Date: Jul. 14, 2023

Related U.S. Application Data
(60) Provisional application No. 63/140,035, filed on Jan. 21, 2021.
(60) Provisional application No. 63/277,796, filed on Nov. 10, 2021.
(60) Provisional application No. 63/291,093, filed on Dec. 17, 2021.

(51) Int. Cl. C12Q 1/52; B01L 3/00; G01N 33/72

(52) U.S. Cl. CPC C12Q 1/52; B01L 3/5023; B01L 3/502715; G01N 33/728; B01L 2200/16; B01L 2300/021; B01L 2300/027; B01L 2300/0654; B01L 2300/069; B01L 2300/0864; B01L 2300/126

(57) ABSTRACT

The present disclosure relates to paper microfluidic devices for use in combination with a viewing box assembly for imaging and rapid identification and quantification of target analytes in a fluid sample that is deposited onto the device such that one or more target analytes in the sample react with one or more diagnostic components on the paper, causing a detectable reaction. The reacted microfluidic device may then be placed inside an opaque viewing box having an internal light source and top panel viewing aperture through which the microfluidic device may be imaged using a mobile electronic device and graphical user-interface for purposes of detecting and quantifying the one or more target analytes. In some embodiments, the microfluidic device includes diagnostic paper and abase. In some embodiments, the microfluidic device includes a filter layer on top of diagnostic paper layer.

### FIELD

The present disclosure generally relates to paper microfluidic devices that may be used in combination with a viewing box assembly for imaging and rapid identification and quantification of target analytes.

### BACKGROUND

Point-of-care (POC) diagnostics are advantageous in many resource-limited settings where healthcare, transportation, and distribution infrastructure may be underdeveloped or underfunded. A main advantage of a POC diagnostic is the ability to diagnose disease or assess health status without the support of a laboratory infrastructure. This increases access, removes the need for sample transport, and substantially reduces the time it takes to obtain diagnostic results. Accordingly, more patients are effectively diagnosed and assessed, enabling more efficient and effective healthcare treatment. Although paper-based diagnostics have been known and used for several years, many paper POC devices lack sufficient accuracy or are economically infeasible due to various factors such as poor limits of detection, high non-specific adsorption, unstable reagents, long analysis time, complex user-technology interface, onerous detection method, and poor sensitivity, among others. Thus, there is a need for an improved paper-based POC device that is sensitive, robust, readily manufactured at relatively low cost, easy to use, and that can be rapidly assessed to provide accurate, quantifiable results without the need for a laboratory infrastructure.

### SUMMARY

The present disclosure generally relates to a rapid paper microfluidic device, optionally comprising a base, that can perform a variety of diagnostic assays on a fluid sample, including but not limited to biological samples (e.g., blood, urine, sputum, saliva, or other bodily fluid) by chemically reacting one or more diagnostic components on the device with one or more corresponding target analytes in the sample. The disclosure also relates to methods of capturing an image of a reacted microfluidic device to generate and quantify diagnostic results corresponding to the physical health and/or condition of a subject from which the biological sample was obtained, or the content (e.g., contaminant content) of other types of fluid samples.

In one aspect, the disclosed technology relates to a recessed microfluidic device, including: a base having an upper surface and a lower surface, the upper surface including at least one recessed fluid transfer channel in fluid communication with a corresponding diagnostic chamber, wherein the diagnostic chamber includes a recessed area substantially surrounded by a raised frame; and diagnostic paper sized to fit within the recessed area of the diagnostic chamber, wherein the diagnostic paper includes one or more diagnostic components provided thereon. In one embodiment, the recessed microfluidic includes three recessed fluid transfer channels fluidically coupled to a common channel entry. In some embodiments, the diagnostic paper is a single layer sheet of hydrophilic, porous paper. In some embodiments, the diagnostic paper is filter paper or chromatography paper.

In some embodiments, the one or more diagnostic components are selected from reagents, dyes, probes, stabilizers, catalysts, anti-coagulants, lysing agents, nanoparticles, diluents, and combinations thereof. In some embodiments, at least one diagnostic component is capable of selectively associating with an analyte selected from aspartate transaminase, alkaline phosphatase, alanine aminotransferase, bilirubin, albumin, total serum protein, glucose, cholesterol, creatine, sodium, calcium, gamma glutamyl transferase, direct bilirubin, indirect bilirubin, unconjugated bilirubin, and lactate dehydrogenase, glucose, blood urea nitrogen, calcium, bicarbonate, chloride, creatinine, potassium, and sodium.

In some embodiments, the recessed microfluidic device further includes a filter in fluid communication with at least one fluid transfer channel, wherein the filter is spaced apart from the diagnostic paper. In some embodiments, the base further includes an extension having an upper surface on which an identifying indicator is provided. In some embodiments, the identifying indicator includes a QR code or barcode.

In another aspect, the disclosed technology relates to a microfluidic device and viewing box assembly, including: a microfluidic device including a single layer of diagnostic paper that includes one or more diagnostic components provided thereon; and a viewing box including a bottom panel, one or more top panels, four or more side panels, and one or more internal light source(s), wherein each top panel includes a viewing aperture; wherein the microfluidic device is configured to fit within the viewing box when the viewing box is assembled.

In some embodiments, the top panel viewing apertures are the only openings through which light may enter the viewing box. In some embodiments, at least the interior of the viewing box is made entirely from solid, opaque material. In some embodiments, an interior surface of the bottom panel includes a position indicator marking to identify a desired placement position of the microfluidic device. In some embodiments, the microfluidic device is a recessed microfluidic device, including a base having an upper surface and a lower surface, the upper surface including at least one recessed fluid transfer channel in fluid communication with a corresponding diagnostic chamber, wherein the diagnostic chamber includes a recessed area substantially surrounded by a raised frame; and the diagnostic paper is sized to fit within the recessed area of the diagnostic chamber. In some embodiments, the microfluidic device includes: a top layer of diagnostic paper and a bottom layer of filter paper; and lamination layers provided on a top surface of the top layer and a bottom surface of the bottom layer, wherein the lamination layers are adhered together and the lamination layers include aligned apertures configured to permit vertical flow of a fluid sample deposited through the top aperture.

In another aspect, the disclosed technology relates to methods of image processing in order to assess the target analyte in a fluid sample. In one embodiment, when detecting for a single analyte, object detection unit may employ object-detection to detect the panels of the microfluidic device and samples therein. In general, exemplary embodiments of the object detection method may include filtering the image. Filtering the image may include removing external anomalies, remove irregularities of the image through shape smoothing, contrast enhancement, etc. Exemplary embodiments of the object detection method may include boundary recognition to determine a largest target area of interest. Such boundary recognition may erode color parameters and background parameters in order to determine true boundaries of the device. Once the largest contour boundary is determined, then specific boundary points may be identified. Boundary points may include vertices or other apex or point defining a contour of a shape of the object boundary. Morphological shape detection may be used to fit any polygon or geometric shape to the boundary points identified by the defined object boundary. The identified shapes may be cut, cropped, scaled, repositioned, or a combination thereof for image processing and color detection. The target area of interest may then be defined as an interior or central portion of the identified shape.

In another aspect, the disclosed technology relates to a method of detecting and quantifying a target analyte in a fluid sample, including the steps of: (a) obtaining a fluid sample; (b) depositing the fluid sample onto a microfluidic device including a single layer of diagnostic paper that includes one or more diagnostic components provided thereon; (c) waiting for a predetermined period of time during which the fluid sample flows to each diagnostic chamber where a reaction occurs between the target analyte in the sample and the one or more diagnostic components; (d) placing the reacted microfluidic device into a viewing box including a bottom panel, one or more top panels, four or more side panels, and one or more internal light source(s), wherein each top panel includes a viewing aperture, and the viewing apertures are the only openings through which light may enter the viewing box; (e) placing a camera of a mobile electronic device over the viewing aperture and capturing an image of the reacted microfluidic device while illuminated by the one or more internal light source(s); (f) transmitting, by the first electronic device, the image to a second electronic device via a communication network; (g) applying, by the second electronic device, one or more object detection models to the image to generate one or more diagnostic results pertaining to the fluid sample; (h) transmitting, by the second device, at least a portion of the diagnostic results to the first electronic device; and (i) displaying, by the first electronic device, a visual representation corresponding to the at least a portion of the diagnostic results on a display of the first electronic device.

In some embodiments, the fluid sample is a biological fluid sample. In some embodiments, the predetermined period of time is about 60 minutes or less from the time the fluid sample is deposited onto the microfluidic device. In some embodiments, the results of step (g) include diagnostic results. In some embodiments, the microfluidic device includes an extension, and the method further includes applying an identifying indicator onto an upper surface of the extension. In some embodiments, the processing of the image includes clustering pixels of the image into a histogram sorted according to color values. In some embodiments, sorting into a histogram according to color values includes: determining an RGB value, modulating the RGB value to a HEX value, and modulating the RGB value to a corresponding color name. In some embodiments, the captured image is received from a mobile device and a graphical user interface (GUI) is displayed at the mobile electronic device.

In some embodiments, the microfluidic device is a recessed microfluidic device, including a base having an upper surface and a lower surface, the upper surface including at least one recessed fluid transfer channel in fluid communication with a corresponding diagnostic chamber, wherein the diagnostic chamber includes a recessed area substantially surrounded by a raised frame; and the diagnostic paper is sized to fit within the recessed area of the diagnostic chamber. In some embodiments, the microfluidic device includes: a top layer of diagnostic paper and a bottom layer of filter paper; and lamination layers provided on a top surface of the top layer and a bottom surface of the bottom layer, wherein the lamination layers are adhered together and the lamination layers include aligned apertures configured to permit vertical flow of a fluid sample deposited through the top aperture.

A variety of additional aspects will be set forth in the description that follows. The aspects can relate to individual features and to combinations of features. It is to be understood that both the foregoing general description and the following detailed description are exemplary and explanatory only and are not restrictive of the broad inventive concepts upon which the embodiments disclosed herein are based.

### BRIEF DESCRIPTION OF THE DRAWINGS

The accompanying drawings, which are incorporated herein and constitute part of this specification, are illustrative of particular embodiments of the present disclosure and do not limit the scope of the present disclosure. The drawings are not to scale and are intended for use in conjunction with the explanations in the following detailed description.

FIG. 1A shows a top view of an example of a base of a microfluidic device in accordance with the present disclosure, wherein the base includes three diagnostic chambers.

FIG. 1B shows a perspective view of the base shown in FIG. 1A.

FIG. 2A shows a top view of an example of a base of a microfluidic device in accordance with the present disclosure, wherein the base includes three diagnostic chambers, each having a substantially centered through hole.

FIG. 2B shows a perspective view of the base shown in FIG. 2A.

FIG. 3A shows a top view of an example of a base of a microfluidic device in accordance with the present disclosure, wherein the base includes three diagnostic chambers and an extension.

FIG. 3B shows a perspective view of the base shown in FIG. 3A.

FIG. 4A shows a top view of another example of a base of a microfluidic device in accordance with the present disclosure, wherein the base includes three diagnostic chambers and an extension.

FIG. 4B shows a perspective view of the base shown in FIG. 4A.

FIG. 5 shows a perspective view of an example of a microfluidic device in accordance with the present disclosure, including a base and single layer diagnostic paper positioned in each a recessed area of each diagnostic chamber.

FIG. 6 shows a perspective view of an example of a viewing box in accordance with the present disclosure, including four side panels, two top panels, and a bottom panel marked with a position indicator.

FIG. 7 shows a perspective view of an example of a first (upper) top panel of a viewing box in accordance with the present disclosure, wherein the upper top panel includes an upper viewing aperture.

FIG. 8 shows a perspective view of an example of a second (lower) top panel of a viewing box in accordance with the present disclosure, wherein the lower top panel includes a lower viewing aperture.

FIG. 9 shows a perspective view of an example of a side panel of a viewing box in accordance with the present disclosure.

FIG. 10 shows a perspective view of another example of a side panel of a viewing box in accordance with the present disclosure.

FIG. 11 shows a perspective view of an example of a bottom panel in accordance with the present disclosure.

FIG. 12 shows an exploded view of a viewing box including a lighting apparatus.

FIG. 13 shows an example of a computing environment including one microfluidic device and a computing device in accordance with the present disclosure.

FIG. 14 shows an example of a computing and networking environment in accordance with the present disclosure.

FIG. 15 shows an example of a process for capturing an image of a microfluidic device to generate diagnostic results in accordance with the present disclosure.

FIG. 16 shows an example image of a microfluidic device having diagnostic chambers arranged in a cloverleaf pattern.

FIG. 17 shows an example image dilation as applied to the image of FIG. 16, in which noise has been filtered from the image.

FIG. 18 shows an example image of a microfluidic device as applied to the image of FIG. 17, in which the image has been further processed and normalized to identify the largest contour boundary of the microfluidic device.

FIG. 19 shows an example image of a microfluidic device, including individually detected contours of the diagnostic chambers of the microfluidic device, cropped and ready to save as a separate image.

FIG. 20 shows an example image of a microfluidic device, including identified bounding areas of diagnostic chambers for color sample detection, extraction, and analysis.

FIG. 21 is an iOS application workflow, as described in Example 2 herein.

FIG. 22 is a flowchart of an image processing workflow, as described in Example 2 herein.

FIG. 23 is a flow chart of a workflow for calculating color spectrum arrays, as described in Example 2 herein.

FIG. 24 is a schematic showing the assembly of another example of a microfluidic device in accordance with the present disclosure, wherein a layer of membrane filter is stacked on top of a layer of diagnostic paper, as described in Example 3 herein.

FIG. 25 is a schematic showing the processing of the microfluidic device of FIG. 24, as described in Example 3 herein.

### DETAILED DESCRIPTION

The following discussion omits or only briefly describes features of the disclosed technology that are apparent to those skilled in the art. It is noted that various embodiments are described in detail with reference to the drawings, in which like reference numerals represent like parts and assemblies throughout the several views. In drawings that depict multiple like components (e.g., multiple diagnostic chambers), a single representative component may be identified by the appropriate reference numeral. Reference to various embodiments does not limit the scope of the claims appended hereto. Additionally, any examples set forth in this specification are intended to be non-limiting and merely set forth some of the many possible embodiments for the appended claims. Further, particular features described herein can be used in combination with other described features in each of the various possible combinations and permutations.

Unless otherwise specifically defined herein, all terms are to be given their broadest possible interpretation including meanings implied from the specification as well as meanings understood by those skilled in the art and/or as defined in dictionaries, treatises, etc. It must also be noted that, as used in the specification and the appended claims, the singular forms “a,” “an” and “the” include plural referents unless otherwise specified, and that the terms “comprises” and/or “comprising,” when used in this specification, specify the presence of stated features, elements, and/or components, but do not preclude the presence or addition of one or more other features, steps, operations, elements, components, and/or groups thereof.

The present disclosure relates to microfluidic devices and methods of use thereof for testing of a fluid sample—e.g., a biological fluid sample obtained from a subject, such as a human or other mammal; or another type of fluid sample, such as a water sample, prepared solution, non-biological sample, and the like. The devices are designed to be usable without the need for a laboratory infrastructure—e.g., in a home, in a mobile unit, or in an out-patient clinical setting, such as a physician's office. In some embodiments, use of the microfluidic device involves depositing a fluid sample onto the device so that the sample flows to single-layer diagnostic paper where the sample chemically reacts with a diagnostic component, resulting in a color change that can be quantified and recorded by taking an image of the reacted device using a viewing box assembly and an application running on a portable electronic device.

Microfluidic Device with Base

In one embodiment, the microfluidic device 1 includes diagnostic paper 13 positioned on a base 2. See FIG. 5. In some embodiments, the diagnostic paper is a single layer sheet of hydrophilic, porous paper. In one embodiment, the diagnostic paper is filter paper or chromatography paper. In some embodiments, the diagnostic paper is formed from a single material. In some embodiments, the diagnostic paper includes or excludes one or more materials selected from nitrocellulose, cellulose acetate, polymer film, cloth, and glass (e.g., borosilicate glass microfiber). Other non-limiting examples of suitable diagnostic papers include the following grades (available from I. W. Tremont): Grade B, Grade B-85, Grade F, Grade C, Grade RG, Grade LL-72, Grade D-23, Grade D-23-TC-1, Grade Fibrous Cellulose Acetate, Grade WT-2500hpc, Grade CFP1, Grade CFP2, Grade CFP 1654, Grade BLOTT, and Grade WT-CFP-PE1.

In some embodiments, one or more of the diagnostic papers is held in place on the base by an adhesive. In general, the adhesive is inert to the paper and to any solutions, reagents, diagnostic components, samples, etc. that may be applied to the paper. Non-limiting examples of suitable adhesives include chemically inert substances such as glue, wax, epoxy, resin, super glue, polyacrylamide, tape, non-absorbent polymer such as polydimethylsiloxane (PDMS), a polyether block amide (e.g., PEBAX®, commercially available from Arkema), a polyacrylate, a polymethacrylate (e.g., poly(methyl methacrylate)), a polyimide, polyurethane, polyamide (e.g., Nylon 6,6), polyvinylchloride, polyester, (e.g., HYTREL®, commercially available from DuPont), polyethylene (PE), polyether ether ketone (PEEK), fluoropolymers such as polytetrafluoroethylene (PTFE), perfluoroalkoxy, fluorinated ethylene propylene, and combinations thereof. In some embodiments, the adhesive is applied to the back of the paper before the paper is placed into the base. In other embodiments, the adhesive is applied to the recessed area of the diagnostic chamber before the paper is placed into the base. In some embodiments, the adhesive covers the entire back surface of the paper, or substantially covers the back surface of the paper so as to securely hold the diagnostic paper in place.

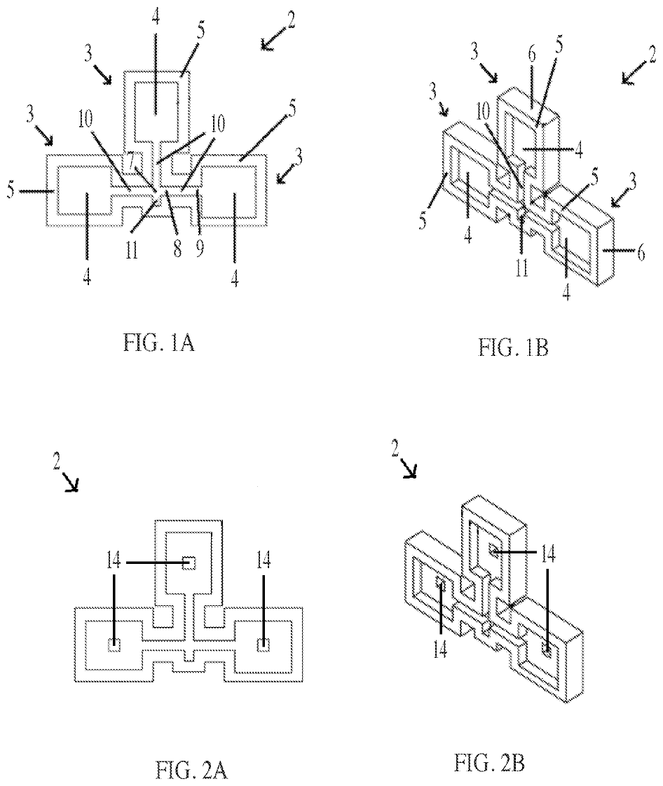

As shown in FIG. 1A and FIG. 1B, the base 2 includes at least one diagnostic chamber 3. In some embodiments, the base 2 includes a plurality of diagnostic chambers, such as 2, 3, 4, 5, 6, or more diagnostic chambers. In some embodiments, the base 2 includes three diagnostic chambers 3, which may be arranged in a cloverleaf pattern, as depicted in FIGS. 1-5. Each diagnostic chamber 3 includes a frame 5 that substantially surrounds a recessed area 4. Each recessed area 4 of each diagnostic chamber 3 is provided with a diagnostic paper 13 that is sized to fit within the length and width dimensions of the recessed area 4. In some embodiments, each recessed area 4 holds a single-layer piece of diagnostic paper 13. The type of diagnostic paper in one diagnostic chamber may be the same as or different from the type of diagnostic paper in one or more other diagnostic chambers.

One or more diagnostic components are provided on each diagnostic paper 13. In some embodiments, the diagnostic components are printed onto the paper. In other embodiments, the diagnostic components are otherwise deposited onto the paper. Non-limiting examples of suitable diagnostic components include: reagents, dyes, probes, stabilizers, catalysts, anti-coagulants (e.g., EDTA or heparin), colorimetric probes, fluorescent probes, lysing agents, nanoparticles, diluents, and combinations thereof. In some embodiments, each diagnostic paper contains one, two, or three diagnostic components. For example, a mixture containing a dye and a reagent that selectively associates with a target analyte may be deposited onto a diagnostic paper. Alternatively, a mixture containing a dye, a stabilizer, and a reagent that selectively associates with a target analyte may be deposited onto a diagnostic paper. Other combinations are contemplated as well, based on the target analyte of interest. The diagnostic components may be provided on the paper either before or after the paper is positioned within a recessed area 4 of a diagnostic chamber 3 of base 2. In some embodiments, each diagnostic paper 13 in the microfluidic device 1 is provided with a different diagnostic component or mixture thereof so as to test for multiple, different analytes within a single fluid sample. References to biological fluid samples herein are provided as non-limiting, representative examples of a fluid sample.

When a target analyte is present in a biological fluid sample that flows onto a diagnostic paper 13 of the microfluidic device 1, the analyte will selectively associate and react with diagnostic components present on the diagnostic paper 13. In some embodiments, such reactions cause a color change, wherein the intensity of the color change corresponds to the concentration of the analyte present in the sample. In some embodiments in which the device includes multiple diagnostic chambers, a user may rapidly test for multiple diseases and/or patient conditions using just one biological fluid sample and one microfluidic device because the different diagnostic papers may contain different diagnostic components that selectively associate with different target analytes.

Each diagnostic chamber 3 of base 2 includes a recessed area 4 substantially surrounded by a frame 5. The top surface of the frame 5 is raised relative to the recessed area 4. An opening in the frame 5 is immediately adjacent a terminal end 9 of a recessed fluid transfer channel 10. As such, each fluid transfer channel 10 is in fluid communication with the recessed area 4 of the corresponding diagnostic chamber 3. The depth of the recessed area 4 may be constant throughout the recessed fluid transfer channels 10 and each diagnostic chamber 3 of the base 2. In some embodiments, the depth of any portion or all of each recessed area 4 and/or each recessed fluid transfer channel 10 is about 1 mm to about 20 mm, about 1 mm to about 15 mm, about 1 mm to about 10 mm, about 1 mm to about 5 mm, about 2 mm to about 20 mm, about 2 mm to about 15 mm, about 2 mm to about 10 mm, or about 2 mm to about 5 mm.

Each fluid transfer channel 10 has an initial end 8 opposite its terminal end 9. A fluid sample deposited onto the microfluidic device 1 at or near the common channel entry 7 will flow horizontally from each initial end 8 of each fluid transfer channel 10 toward each terminal end 9 of each fluid transfer channel 10 to reach the diagnostic paper 13 of each corresponding diagnostic chamber 3. In some embodiments, an overflow well 11 is fluidically connected to the initial end 8 of the fluid transfer channel 10. In a microfluidic device 1 containing more than one diagnostic chamber 3 and corresponding fluid transfer channel 10, each initial end 8 of each fluid transfer channel 10 intersects at a common channel entry 7. In some embodiments, the overflow well 11 is fluidically connected to the common channel entry 7. In general, the overflow well 11 serves to retain excess sample that has been deposited onto the device in order to prevent overflow or run-off.

In some embodiments, the frame 5 of the diagnostic chamber 3 is formed from a solid material. Non-limiting examples of suitable solid materials include plastics such as acrylic polymers, acetal resins, polyvinylidene fluoride, polyethylene terephthalate, polytetrafluoroethylene (e.g., TEFLON®), polystyrene, polypropylene, other polymers, thermoplastics, glass, ceramics, metals, and the like, and combinations thereof. In general, the selected solid materials are inert to any solutions/reagents that will contact them during use or storage of the device. The base 2 may be fabricated by various means, partly dependent upon the chosen materials. Any known fabrication method appropriate to the selected solid material(s) may be employed including, but not limited to, machining, die-cutting, laser-cutting, stereolithography, chemical/laser etching, integral molding, lamination, and combinations thereof. The base can be integrally formed as a single, unitary piece. Alternatively, a plurality of separate parts (e.g., separate diagnostic chambers and fluid transfer channels, separate units of integrally formed single piece diagnostic chambers and fluid transfer channels, etc.) can be attached together to collectively form the base.

In some embodiments, each fluid transfer channel 10 is recessed to the same depth as each of the recessed areas 4 of the corresponding diagnostic chambers 3. In some embodiments, a bottom surface of the base is smooth and extends across a single plane such that the base is level when seated on a larger surface, such as a table or a bottom panel 21 of the viewing box 30, discussed below. In some embodiments, the top surface of each frame 5 is smooth and in the same plane.

In some embodiments, each fluid transfer channel 10 has substantially the same length, such that the biological fluid sample flows from the point at which the sample is deposited toward each diagnostic paper 13 in approximately the same amount of time and in approximately the same volume. In some embodiments, the fluid transfer channels 10 have different lengths. In some embodiments, one or more fluid transfer channels 10 may have a length of about 2 mm to about 40 mm, about 2 mm to about 30 mm, about 2 mm to about 20 mm, about 2 mm to about 15 mm, about 2 mm to about 10 mm, about 2 mm to about 5 mm, about 4 mm to about 40 mm, about 4 mm to about 30 mm, about 4 mm to about 20 mm, about 4 mm to about 15 mm, or about 4 mm to about 10 mm. The length of a fluid transfer channel 10 is measured from its terminal end 9 (adjacent the edge of the recessed area 4 of a diagnostic chamber 3—i.e., where the opening of the frame 5 is located) to its initial end 8, which is at the center of the common channel entry 7 in embodiments comprising more than one diagnostic chamber 3.

The recessed area 4 of each diagnostic chamber 3 may have the same or different length and width dimensions as one or more recessed areas of other diagnostic chambers. In some embodiments, a length or width of a recessed area 4 of a diagnostic chamber 3 may be about 5 mm to about 20 mm, such as about 7 mm to about 15 mm, or about 8 mm to about 11 mm. The length and width of a recessed area 4 of a diagnostic chamber 3 may the same or different. The overall dimensions of the diagnostic chamber 3, including both the recessed area 4 and the frame 5, are necessarily larger than the overall dimension of the recessed area 4. In some embodiments, the frame 5 has a consistent width of about 0.5 mm to about 8 mm, about 1 mm to about 6 mm, or about 2 mm to about 4 mm.

In embodiments comprising a plurality of diagnostic chambers, the diagnostic chambers may be arranged in variety of configurations—e.g., as a cloverleaf, an “X,” a “Y,” etc. In a cloverleaf configuration, for example, as depicted in FIGS. 1-5, a first diagnostic chamber 3 and fluid transfer channel 10 and a second diagnostic chamber 3 and fluid transfer channel 10 extend in opposite directions, and a third diagnostic chamber 3 and fluid transfer channel 10 extends in a substantially perpendicular direction therefrom, and the initial ends of the fluid transfer channels are in fluid communication at the common entry channel point. Each fluid transfer channel 10 is configured to direct the horizontal flow of a biological fluid sample to the diagnostic paper 13 positioned in the corresponding diagnostic chamber 3. In general, once the fluid reaches the diagnostic paper 13, the fluid is propelled by capillary action.

In some embodiments, the bottom surface of the microfluidic device 1 may be flat with no recessed areas or diagnostic chambers, or alternatively may be a mirror of the front surface, having at least one diagnostic chamber and fluid transfer channel, or a matching number of diagnostic chambers and fluid transfer channels, or may have a mix of some diagnostic chambers and some flat areas. For example, a microfluidic device 1 in a cloverleaf shape having a top surface with three recessed diagnostic chambers and three fluid transfer channels in fluid communication with a common channel entry 7, may have a bottom surface with three recessed diagnostic chambers and fluid transfer channels, effectively doubling the number of diagnostic chambers of the microfluidic device 1.

As shown in FIG. 2A and FIG. 2B, the base 2 may include a through hole 14 that fully extends through the recessed area 4 of at least one diagnostic chamber 3. The through hole 14 may have any of a variety of shapes, such as square, circle, polygon, etc. The through hole 14 is generally small, with the largest dimension (length, diameter, etc.) being about 0.5 mm to about 10 mm, such as about 1 mm to about 3 mm, about 1 mm to about 5 mm, about 1 mm to about 7 mm, about 1 mm to about 9 mm, about 2 mm to about 5 mm, or about 2 mm to about 7 mm, or about 2 mm to about 9 mm. In some embodiments, a biological fluid sample may be applied through the through hole 14 to the diagnostic paper 13 in the recessed area 4 of a diagnostic chamber 3—e.g., projected onto the underside of the diagnostic paper 13 via pipette through the through hole 14 in the underside of the base 2. In such an embodiment, the fluid sample would not need to flow through a fluid transfer channel 10 to reach the diagnostic paper 13. This configuration may be useful when there is only a small amount of fluid sample available, and depositing the sample into a fluid transfer channel 10 or at the common channel entry 7 might not allow a sufficient amount of the sample to reach the diagnostic chamber(s) 3. Alternatively, depositing the fluid sample directly onto the top surface of the diagnostic paper 13 could be useful when only a small amount of fluid sample is available.

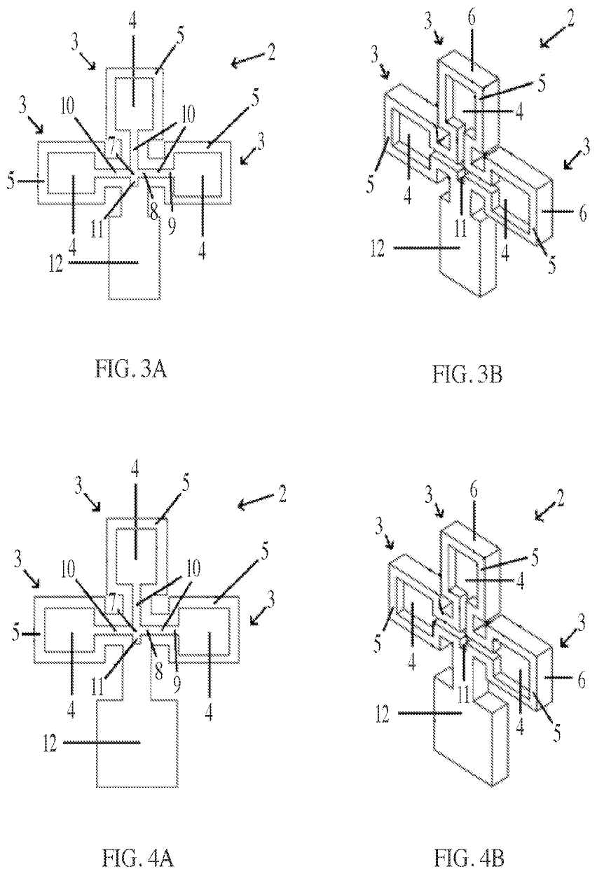

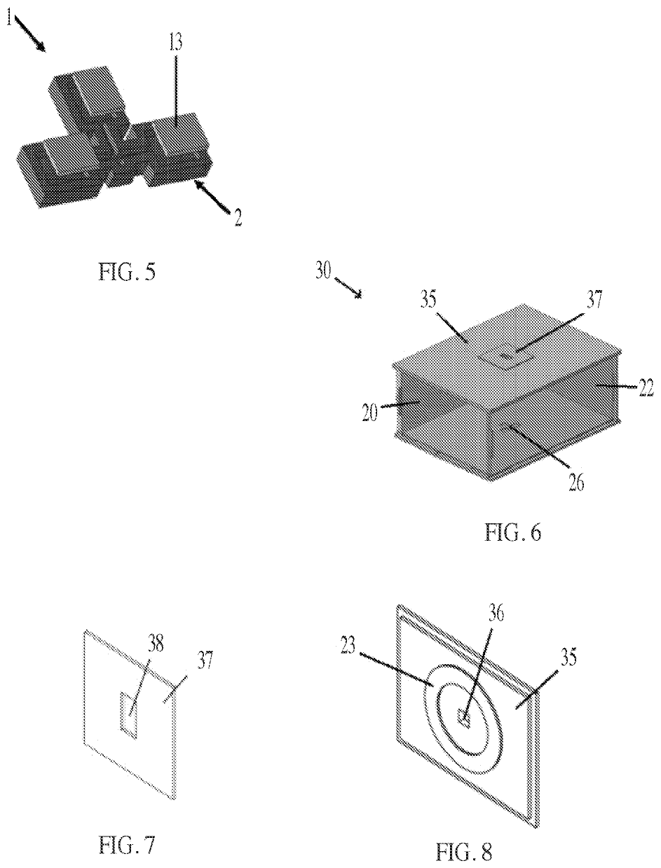

As shown in FIG. 3A, FIG. 3B, FIG. 4A, and FIG. 4B, the base 2 may include an extension 12, which may extend outwardly in a similar manner as the fluid transfer channels 10 and diagnostic chambers 3. The extension 12 may be of the same or similar size as a diagnostic chamber 3. For example, the length and width of the extension 12 may be smaller than, the same as, or large than the corresponding length and width of a diagnostic chamber 3. In some embodiments, the extension 12 has a length and/or width of about 5 mm to about 40 mm, about 5 mm to about 30 mm, about 5 mm to about 20 mm, about 5 mm to about 10 mm, about 10 mm to about 40 mm, about 10 mm to about 30 mm, or about 10 mm to about 20 mm. In some embodiments, as shown in FIG. 3A and FIG. 3B, for example, the length of the extension 12 is greater than its width.

The extension 12 may be configured to have a smooth, flat upper (top) surface suitable for an identifying indicator to be printed, etched, adhered, or otherwise displayed thereon. In various embodiments, an identifying indicator may include an optical pattern. An optical pattern refers to an optical representation of data presented in a sequence or other pattern which can be read by an optical sensor. Examples of optical patterns include, without limitation, a bar code, Quick Response (QR) code, data codes, and/or the like. When an optical sensor (e.g., a camera of an electronic device) scans an identifying indicator, it may detect the representation of data.

When scanned, the identifying indicator provides identifying information related to the microfluidic device 1, the fluid sample, and the source of the fluid sample (e.g., patient data regarding the patient from which a biological fluid sample was obtained). Non-limiting examples of identifying information include: subject name, subject birth date, target analyte(s), type of assay(s), date of assay(s), type of fluid sample, etc. The extension 12 may also serve to stabilize the microfluidic device 1, providing a counterweight to one or more diagnostic chambers. The extension 12 may also provide the user with a convenient means for holding the microfluidic device 1 without touching the diagnostic chambers or fluid transfer channels, thus avoiding potential contamination of the diagnostic chamber(s), sample, or assay(s). In some embodiments in which the microfluidic device does not include an extension 12, a user may hold the microfluidic device 1 along its sides 6.

Microfluidic Device without Base

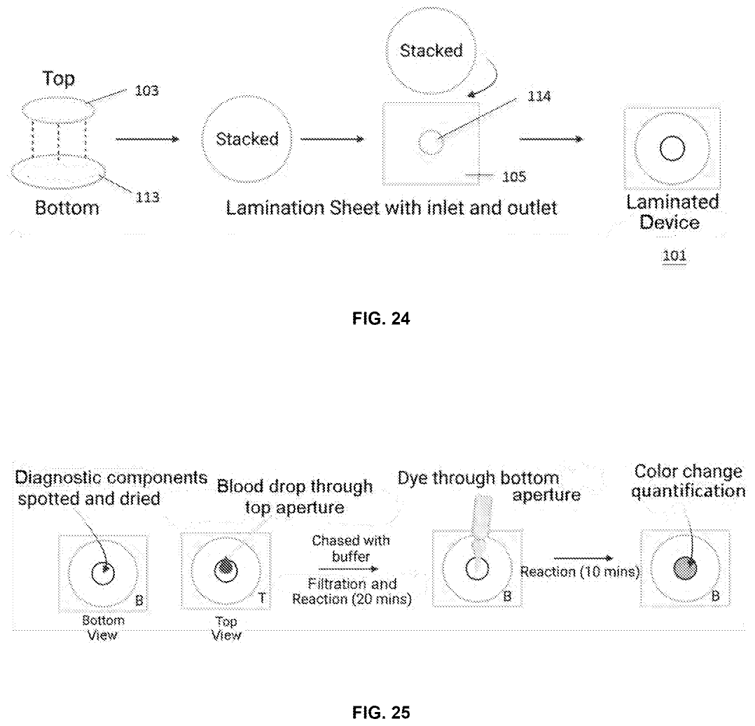

In another embodiment, microfluidic device 101 comprises diagnostic paper 113 and filter paper 103, wherein the device does not include a base (such as base 2 described above). In some such embodiments, as depicted in FIG. 24, the microfluidic device includes only two layers: a top filter layer and a bottom diagnostic layer. The top filter layer may be a single layer of filter paper, such as a plasma separation membrane (e.g., D23, TC-1, MF1, F5, combinations thereof, etc.), that is capable of filtering out components that could interfere with the diagnostic reaction. D23 is a whole blood separation media available from I. W. Tremont and is made from borosilicate glass media, 0.5 mm thick. D23-TC-1 is a whole blood separation media, thin caliper available from I. W. Tremont and is made from borosilicate glass media, 0.375 mm thick. MF1 is a glass fiber filter typically used for whole blood volumes and is available from Cytiva Life Sciences. F5 is a fast-flow single layer matrix membrane available from Cytiva Life Sciences. The bottom diagnostic layer may be a diagnostic paper material as discussed above. For instance, when the fluid sample is whole blood, the filter layer will remove red blood cells from the sample, allowing other target analyte-containing blood component(s), such as serum, to vertically pass through the top filter layer into the bottom diagnostic layer for reaction with the diagnostic component(s).

The top and bottom layers are aligned and stacked together, as depicted in FIG. 24. In some embodiments, the top and bottom layers have the same dimensions, such as the same diameter, length, width, etc. The stacked layers may then be laminated by applying a lamination sheet 105 (or lamination layer) on top of the top layer. The lamination sheet includes an inlet aperture 114 (shown as a circular cutout or hole in FIG. 24) through which the fluid sample is deposited. Optionally, the two paper layers may be completely laminated by providing a second lamination sheet 105 under the bottom layer. The second lamination sheet 105 has an outlet aperture (not shown), and the two lamination sheets and their respective apertures are aligned with each other. In some embodiments, the inlet and outlet apertures are centrally positioned, as depicted in FIG. 24. The dimensions of the lamination sheet(s) may be larger than those of the stacked layers, as depicted in FIG. 24. In this manner, outer portions of the lamination sheets adhere to each other. The inlet and outlet apertures may have any desired shape (circle, square, oval, etc.), preferably the same shape. In some embodiments, the maximum diameter of the inlet/outlet aperture is about 3 mm to about 10 mm, about 3 mm to about 7 mm, or about 4 mm to about 6 mm. Advantageously, lamination applies pressure to the paper layers, increasing the flow rate of the fluid sample through the layers and thus achieving a more rapid diagnostic assay.

Although FIG. 24 shows a device having a single set of stacked layers with a single top aperture for sample deposition, the present disclosure contemplates devices having a plurality of apertures in the laminated layer(s) that would allow for multiple deposits of the same or different fluid samples to be deposited onto the device so that multiple assays could be conducted simultaneously with a single device. For example, such a device could include an array of 2, 3, 4, 5, 6, 7, 8 or more laminated stacked layers, each having an aperture through which fluid sample could be deposited. In one such embodiment, a plurality of the devices having the configuration shown in FIG. 24 could be arranged adjacent one another in a 2×2 (i.e., 4 devices), 3×2 (i.e., 6 devices), 4×2 (i.e., 8 devices), or any other like arrangement. In some embodiments, each microfluidic device has a width and/or length of about 1.5 cm to about 5 cm, such as about 2 cm to about 3 cm. Accordingly, for illustrative purposes only, an array of 4×2 optionally laminated microfluidic devices could have an overall width of about 3 cm to about 10 cm, such as about 4 cm to about 6 cm; and an overall length of about 6 cm to about 20 cm, such as about 8 cm to about 12 cm.

In some embodiments, the top filter layer and bottom diagnostic paper layer are stacked together by placing one on top of the other in the absence of any adhesive therebetween. Features of the diagnostic paper (e.g., single layer, type of material, etc.) discussed above in relation to a microfluidic device with a base are equally applicable to the diagnostic paper discussed in this section in relation to a microfluidic device without a base. Features of the one or more diagnostic components (e.g., method of application onto the diagnostic paper, types and combinations of components, timing of applying the components, etc.) and reactions with target analytes discussed above in relation to a microfluidic device with a base are equally applicable to the diagnostic paper discussed in this section in relation to a microfluidic device without a base.

Viewing Box

As shown in FIG. 6, the present disclosure also relates to a viewing box 30 configured to enhance the imaging of the microfluidic device 1 after completion of the assay(s). The viewing box 30 may be made from any solid material that does not permit any light to pass through. In some embodiments in which at least the interior of the viewing box 30 is made entirely from one or more solid materials, other types of materials may be added to the viewing box 30, provided that any such added materials do not degrade the light-blocking function of the viewing box 30 itself. Non-limiting examples of suitable solid materials include cardboard, acrylic, glass (e.g., painted glass), ceramics, metals, and combinations thereof. The viewing box 30 is comprised of a plurality of panels, including at least a bottom panel, a top panel, and four side panels. Any two or more of the panels (e.g., 2, 3, 4, or 5 panels) may be formed as a unitary piece. Alternatively, the panels may be separate pieces that are connected together to form the viewing box 30.

FIG. 6 depicts an assembled viewing box 30 having a bottom panel 21, two long side panels 20, two short side panels 22, a lower top panel 35, an upper top panel 37. The viewing box 30 of FIG. 6 is shown with see through sides for illustrative purposes in order to reveal the features within the viewing box 30. As described above, the side panels may be made from a solid, opaque material. The side panels together form the side walls of the box assembly. Each side panel has a length, height, and width (thickness). In some embodiments, all of the side panels have approximately the same height. In some embodiments, all of the side panels have approximately the same thickness. In some embodiments, two opposing side panels have the same length, which is greater than the length of the other two opposing side panels. In other embodiments, all of the side panels have approximately the same length. In some embodiments, the length of one or more side panels is about 70 mm to about 400 mm, such as about 150 mm to about 300 mm. In some embodiments, the height of one or more side panels is about 50 mm to about 150 mm, such as about 80 mm to about 120 mm. In some embodiments, the thickness of one or more side panels is about 1 mm to about 6 mm, such as about 2 mm to about 4 mm.

Each top panel 35, 37 and bottom panel 21 has a length, height (thickness), and width. The length and width of the bottom panel 21 may be the same as that of at least one of the top panels 35, 37. In some embodiments, the top and bottom panels all have approximately the same thickness—e.g., about 1 mm to about 6 mm, such as about 2 mm to about 4 mm. In some embodiments, the top and bottom panels all have approximately the same length—e.g., about 50 mm to about 200 mm or about 50 mm to about 100 mm. to about 250 mm to about 300 mm to about 350 mm to about 400 mm. In some embodiments, the top and bottom panels all have approximately the same width—e.g., about 150 mm to about 200 mm to about 250 mm to about 300 mm.

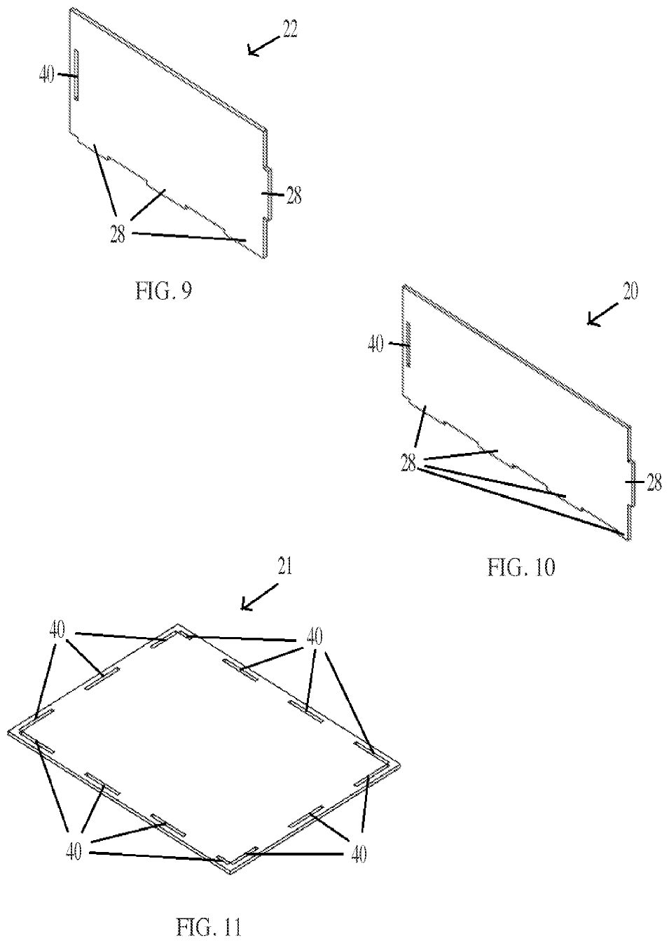

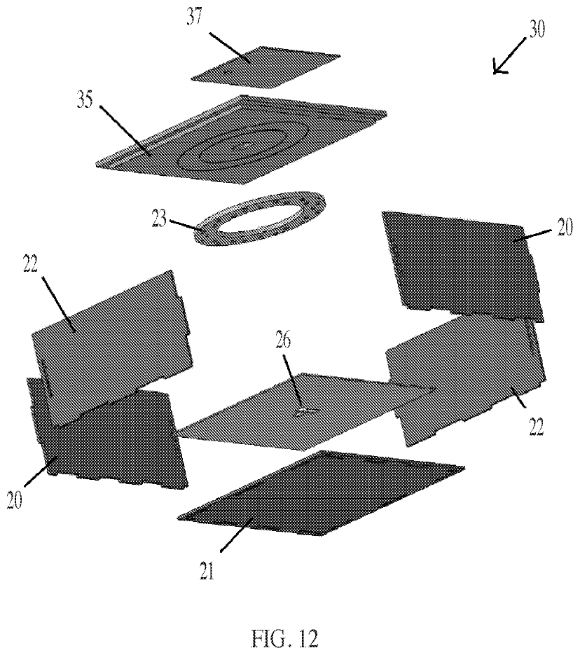

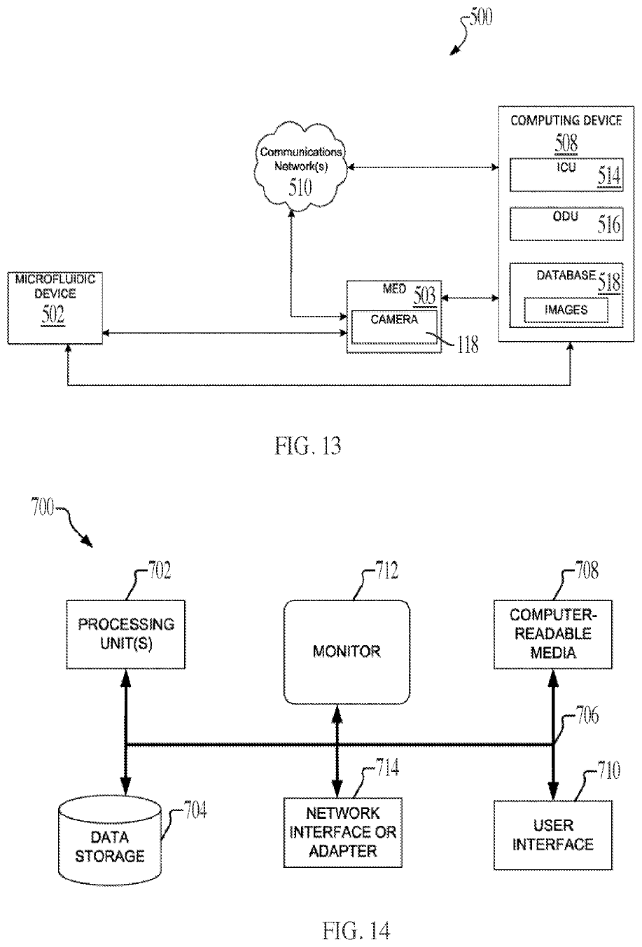

In some embodiments, the side panels 20, 22 are configured to securely connect with the bottom panel 21. For example, as shown in FIGS. 6 and 9-11, the short side panels 22 and long side panels 20 are attached to the bottom panel 21 via a series of fins 28 protruding from the bottom edges of the side panels, wherein the fins fit within a corresponding series of slits (openings) 40 along the outer perimeter of the bottom panel 21. Alternatively, the side panels 20, 22 may be configured to securely connect with the bottom panel 21 via a series of slits 40 along the bottom edge of each side panel 20, 22, wherein a corresponding series of fins 28 protruding from the outer perimeter of the bottom panel 21 are configured to fit within the slits 40. Other viewing box 30 configurations are contemplated as being suitable for use with the present disclosure. In general, the viewing box 30 is designed to block light from outside sources (e.g., ambient light) that could disrupt the clarity of the image taken of a reacted microfluidic device 1 positioned inside the viewing box 30.

In some embodiments, the bottom panel 21 includes a position indicator 26 to identify where the microfluidic device 1 should be placed for optimal imaging within the viewing box 30. See FIG. 6. In general, the position indicator may be a visible marking, such as a silhouette corresponding to a microfluidic device 1, a line, a rectangle, an arrow, etc. In some embodiments, the position indicator 26 comprises a recess within which a microfluidic device 1 may fit, optionally snugly fit. In general, the position indicator 26 is centrally positioned on the upper (interior) surface of the bottom panel 21. In some embodiments, the position indicator 26 is provided on a panel that is positioned on the bottom panel 21 and fits within the viewing box 30. To use a viewing box 30 with the disclosed microfluidic device, a user may position a reacted microfluidic device 1 (i.e., after completion of the desired assays(s)) on the position indicator 26 and then close the one or more top panels of the viewing box 30 so that all panels are in place.

Each top panel includes a viewing aperture through which a user may view a microfluidic device 1 seated on the position indicator 26 of the bottom panel 21. As depicted in FIG. 6, the viewing box 30 includes two top panels: an upper top panel 37 placed over a lower top panel 35, wherein the upper top panel 37 is smaller than the lower top panel 35. The upper top panel 37 includes an upper viewing aperture 38. The lower top panel 35 includes a lower viewing aperture 36. The upper viewing aperture 38 is larger than the lower viewing aperture 36. In general, in embodiments having more than one top panel, at least two top panels are configured such that one is larger than the other. An upper top panel may be positioned over a lower top panel. An upper top panel may be smaller than a lower top panel, or vice versa. In one embodiment, the lower top panel may contact the top edges of all four of the side panels to serve as the structural top of the viewing box 30. In one embodiment including a lower top panel having a lower viewing aperture and an upper top panel having an upper viewing aperture, the apertures are of different sizes—i.e., the upper viewing aperture may be larger than the lower viewing aperture, or the upper viewing aperture may be smaller than the lower viewing aperture. In embodiments having more than one top panel, the viewing apertures must be aligned such that a user can see through them, and the viewing aperture of each panel may be differently sized to narrow or focus the field of view as seen through the viewing apertures.

In some embodiments, the viewing aperture is centrally positioned in the top panel. Each viewing aperture fully extends through its top panel. A viewing aperture may have any of a variety of shapes, such as square, circle, polygon, etc. In some embodiments, the largest dimension (length, diameter, etc.) of a viewing aperture is about 0.5 mm to about 30 mm, about 1 mm to about 5 mm, about 1 mm to about 10 mm, about 1 mm to about 15 mm, about 1 mm to about 20 mm, about 1 mm to about 25 mm, about 3 mm to about 10 mm, about 3 mm to about 15 mm, about 3 mm to about 20 mm, about 3 mm to about 25 mm, about 5 mm to about 40 mm, about 5 mm to about 10 mm, about 5 mm to about 20 mm, about 5 mm to about 30 mm, about 10 mm to about 20 mm, about 10 mm to about 30 mm, or about 10 mm to about 40 mm. In some embodiments, a transparent film or layer may be provided over one or more of the viewing apertures. The viewing apertures 36, 38 of the top panels need not be centered in the top panels themselves, and in some embodiments may be off-center, so long as they are aligned with one another and the position indicator 26 of the bottom panel 21.

FIG. 12 shows an exploded view of a viewing box 30. From top to bottom, shown are an upper top panel 37 and a lower top panel 35. In some embodiments, the interior surface of the innermost top panel (depicted in FIG. 12 as lower top panel 35) is equipped with one or more internal light source(s) 23. In the depicted embodiment, walls are formed from long side panels 20 and short side panels 22. The position indicator 26, shown in FIG. 12 as a separate panel, rests on top of the bottom panel 21. Non-limiting examples of suitable light sources include light emitting diodes (LEDs), condensed fluorescent lights (CFL), halogen lights, and other similar light sources. The lights may be powered internally by battery, externally by power cord or optional solar panels, or other similar power options. The one or more internal light source(s) may be the sole source of light that illuminates the microfluidic device 1 inside the viewing box 30 when a handheld device is positioned over the viewing aperture(s). As such, the one or more internal light source(s) may provide consistent lighting and thus consistent imaging conditions. This is highly advantageous because, assessment of a color change of a reacted microfluidic device can be challenging to visually or even electronically discern when exposed to changing or ambient lighting. Accordingly, the viewing box 30 can provide a type of “portable dark room” in which to view and image microfluidic devices after assay completion. In some embodiments, the one or more internal light source(s) is provided at least along the underside (interior surface) of the innermost top panel (e.g., lower top panel 35), such as in one or more locations along the perimeter of such interior surface. In some embodiments, the one or more internal light source(s) 23 may be provided on the interior surface of one or more of the top, bottom, and side panels to provide the desired amount of illumination. In some embodiments, the one or more internal light source(s) 23 is a comprised of a single light (e.g., in the form of a ring) or the one or more internal light source(s) 23 is a plurality of lights. Once the microfluidic device is in place on the position indicator 26 inside the viewing box 30, the viewing box 30 is closed, and the interior light source is illuminated. A mobile electronic device or imaging device, such as a handheld mobile device with a camera, may then be situated over the uppermost viewing aperture so as to take an image of the microfluidic device 1 for subsequent processing in order to quantify the amount of target analyte(s) present in the tested fluid sample.

Although a specific configuration of the viewing box is provided herein, exemplary embodiments are not so limited. For example, a rectangular or square cube is disclosed having a bottom, top, and four sides. However, other shapes are contemplated herein, such as cylindrical, domed, etc. Exemplary embodiments of the viewing box comprise one or more top surfaces, one or more bottom surfaces, and one or more side surfaces to define an enclosed space. The various surfaces of the viewing box may be of different shapes. Various sidewalls are contemplated herein. For example, four separate planar sides may be used and coupled together. A single integrated side may be bent to form a three or four or other multiple-sided enclosure. A single integrated side may be curved to form cylindrical, oval, ovoid, or curved enclosure. In an exemplary embodiment, the top surface may comprise a planar portion to support and/or position a mobile electronic device. In an exemplary embodiment, the bottom surface may comprise a planar portion to support and/or position a microfluidic device. Exemplary embodiments of the top surface, bottom surface, and one or more side surfaces may comprise a plurality of planar component portions configured to interconnect. The interconnection may be through mated slits/projections, surfaces and flanges, other mated surfaces, or a combination thereof. The viewing box may comprise an aperture for taking an image of the microfluidic device, or a portion thereof.

Computing System and Computing Architecture

The disclosed technology also relates to a computing system and computing architecture for use in connection with the disclosed microfluidic device and viewing box assembly. FIG. 13 illustrates an example computing environment 500 comprising a microfluidic device 502, a computing device 508, and a mobile electronic device 503 (illustrated as MED 503), all of which may be deployed with the computing environment 500 to enable or otherwise automate performing a variety of diagnostic assays on a fluid sample. The mobile electronic device 503 and the computing device 508 may be functionally and communicatively connected via a communications network 510, which may be an IP-based telecommunications network, the Internet, an intranet, a local area network, a wireless local network, a content distribution network, or any other type of communications network, as well as combinations of networks. Alternatively, the microfluidic device 502, mobile electronic device 503, and computing device 508 may be functionally and communicatively connected according to a local arrangement, in which such devices directly interact with one another, for example via a hardline or wireline, or other physical and/or optical mechanism that enables operative communication, function, and data transfer.

As used herein, a “computing device” refers to a device that includes a processor and memory. Each device may have its own processor and/or memory, or the processor and/or memory may be shared with other devices as in a virtual machine or container or network arrangement. The memory will contain or receive programming instructions that, when executed by the processor, cause the electronic device to perform one or more operations according to the programming instructions.

As used herein, the terms “memory,” “memory device,” “data store,” “data storage facility” and the like each refer to a non-transitory device on which computer-readable data, programming instructions or both are stored. Except where specifically stated otherwise, the terms “memory,” “memory device,” “data store,” “data storage facility” and the like are intended to include single device embodiments, embodiments in which multiple memory devices together or collectively store a set of data or instructions, as well as individual sectors within such devices.

As used herein, the terms “processor” and “processing device” each refer to a hardware component of an electronic device that is configured to execute programming instructions. Except where specifically stated otherwise, the singular term “processor” or “processing device” is intended to include both single-processing device embodiments and embodiments in which multiple processing devices together or collectively perform a process.

The computing devices described herein are non-conventional systems at least because of the use of non-conventional component parts and/or the use of non-conventional algorithms, processes, and methods embodied, at least partially, in the programming instructions stored and/or executed by the computing devices. For example, exemplary embodiments may use configurations of and processes involving a unique microfluidic device as described herein, configurations of and processes involving a unique viewing box, unique processes and algorithms for object detection for detection and extraction of panels or areas of interest of the panels for analysis, unique configurations and processes of color processing to determine diagnostic results, or combinations thereof. The systems and methods described herein also include POC diagnostics that are unique from conventional systems and methods, even as compared to those diagnostic devices used within laboratory settings. Exemplary embodiments may be used to effectively diagnose and assess patients at the point of care, within a shortened turnaround time, without transporting the fluid samples large distances, enabling more efficient and effective healthcare treatment. Exemplary embodiments described herein include paper-based diagnostics that may be unique in providing sufficient accuracy and/or are economically feasible. Exemplary embodiments described herein include systems and methods of an improved paper-based POC device that is sensitive, robust, readily manufactured at relatively low cost, easy to use, and that can be rapidly assessed to provide accurate, quantifiable results without the need for a laboratory infrastructure. Exemplary embodiments described herein include unique and beneficial image processing techniques and algorithms that permit the diagnostic system to be used with any frame or microfluidic device shape according to embodiments described herein without preprograming or entry of the microfluidic shape into the system before detection and diagnostic. Although exemplary benefits are provided herein, a person of skill in the art would appreciate that any combination of the benefits provided may be realized without departing from the full scope of the disclosure. Therefore, no single benefit, component, or attribute is necessary to the practice of the invention.

In the illustrated computing environment 500, the microfluidic device 502 may be a microfluidic device, as illustrated in FIG. 1A, FIG. 2A, FIG. 3A, or FIG. 4A. The computing device 508 may be a processing device, processor, processors, mobile device, server computing device, and/or any other computing device capable of processing and/or interpreting programming instructions.

In some embodiments, computing device 508 includes an image-capturing unit 514 (illustrated as ICU 514). As used herein, an “image-capturing unit” refers to any device capable of optically viewing an object and converting an interpretation of that object into electronic signals. One such example of an ICU is a camera. An ICU may capture or otherwise obtain one or more images of the data output by and/or at the microfluidic device 502. In some embodiments, the ICU 514 may capture images of the entirety of one or more of the microfluidic device 502. As described herein, an image-capturing unit 514 may be additionally or alternatively incorporated into a mobile device MED 503. The computing device 508 may therefore include an image receiving unit to retrieve image data from an image-capturing unit of either the computing device 508 and/or the MED 503. The computing device may therefore not necessarily include an ICU 514.

The computing device 508 also includes an object-detection unit (ODU) 516 (illustrated as ODU 516) that executes various machine-learning models to process the data (e.g., images) captured at the microfluidic device 502.

As used herein, a “machine learning model” or “model” each refers to a set of algorithmic routines and parameters that can predict an output(s) of a real-world process (e.g., to provide diagnostic results of a processed fluid sample, etc.) based on a set of input features, without being explicitly programmed. A structure of the software routines (e.g., number of subroutines and relation between them) and/or the values of the parameters can be determined in a training process, which can use actual results of the real-world process that is being modeled. Such systems or models are understood to be necessarily rooted in computer technology, and in fact, cannot be implemented or even exist in the absence of computing technology. While machine learning systems utilize various types of statistical analyses, machine learning systems are distinguished from statistical analyses by virtue of the ability to learn without explicit programming and being rooted in computer technology.

In some embodiments, the computing device 508 may include a data store 518 for storing and retrieving captured images. Although the data store 518 of FIG. 13 is depicted as being located within the computing device 508, it is contemplated that the data store 518 may be located external to the computing device 508, for example, at a remote location, and may communicate with the computing device 508 via the communications network 510. Additionally, although the object-detection unit 516 is illustrated as being located within the computing device 508, it is contemplated that the object-detection unit 516 may be located directly within the microfluidic device 502 as a form of executable instructions defining the algorithm(s) (e.g., as a software plug-in).

Referring generally again to FIG. 13, a user may interact with the mobile electronic device 503 to initiate a process through which a variety of diagnostic assays may be performed on a fluid sample. More specifically, and as will be described in further detail below, the mobile electronic device 503 may be used to capture information corresponding to a particular patient, information corresponding to a fluid sample of the patient, and automatically initiate various diagnostic assay processes. The mobile electronic device 503 may be a portable electronic device such as, for example, a laptop personal computer, mobile device, mobile phone, tablet device, and/or other remote processing device capable of implementing and/or executing processes, software, applications, etc., that includes network-enabled devices and/or software, for communication over the communications network 510 (e.g., internet). Additionally, the mobile electronic device 503 may include one or more processors that process software or other machine-readable instructions and may include a memory to store the software or other machine-readable instructions and data. The mobile electronic device 503 may further include a microphone and/or camera (or other optical sensor) that can be used to capture images and/or image data, such as images of the microfluidic device 502.

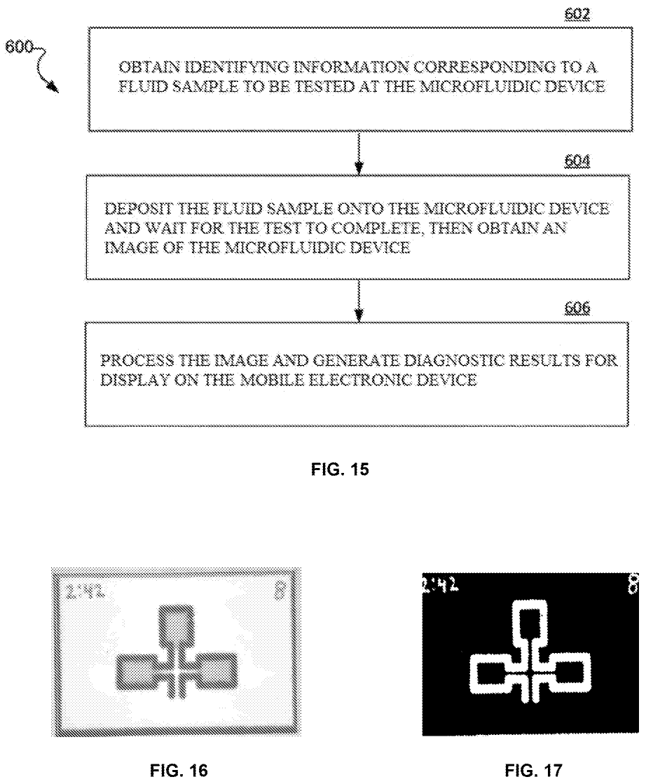

FIG. 15 illustrates a flowchart of one example process 600 for processing diagnostic assay data and automatically generating diagnostic results. The process 600 describes operations performed in connection with the microfluidic device described herein and in particular FIG. 1A-4B. In one specific example, the process 600 may represent an algorithm that can be used to implement one or more software applications that direct operations of a various components of the computing environment 500.

As illustrated, process 600 begins at 602, with obtaining patient information corresponding to a particular patient who has provided a biologic sample for diagnostic testing, or a patient who is interested in providing a biologic sample at a microfluidic device for diagnostic testing. In one specific example and with reference to FIG. 3A, a mobile electronic device 503 may scan or otherwise capture an image of an identifying indicator on a microfluidic device. As explained above, an identifying indicator may be a barcode, a QR code, or other unique identifier. Upon such scanning or capture, the mobile electronic device 503 may obtain certain information, such as patient information. In other embodiments, scanning an identifying indicator may cause the mobile electronic device 503 to prompt a user for certain information. This information may include, without limitation, patient information. In various embodiments, the information that a mobile electronic device 503 obtains and/or prompts a user for may be dependent on the identifying indicator. For instance, an identifying indicator may uniquely identify a microfluidic device as being associated with a particular diagnostic test, and may prompt a user for particular information relating to that diagnostic test. The mobile electronic device 503 may transmit the patient information to the computing device 508.

At 604, an image corresponding to the microfluidic device containing the biologic sample of the particular patient is obtained. Stated differently, images of the fluid sample obtained at the microfluidic device 502 may be captured by the mobile electronic device 503. In an exemplary embodiment, a fluid sample may be deposited onto a microfluidic device according to embodiments described herein. For example, a fluid sample may be applied directly to diagnostic paper 13, into the through hole 14 of recessed area 4 of base 2, or at the initial end 8 or, or along the transfer channel 10, or at the common channel entry 7 before traversing one or more of the transfer channel(s). Once the fluid sample contacts the diagnostic paper, a reaction may occur, and the test may complete. In an exemplary embodiment, the microfluidic device may be positioned or enclosed within the viewing box 30. The microfluidic device may be positioned in relationship to the position indictor 26. The viewing box may be enclosed to reduce or eliminate ambient light to the microfluidic device. A light source may be used to generate a light within the microfluidic device for capturing an image. An image of the microfluidic device may be obtained through one or more image capture units, such as camera of 118 of MED 503 or ICU 514 of computing device 508. In one specific example, an image of the diagnostic chamber may be captured. In another example, a complete image of a microfluidic device may be captured. The image may be captured by the mobile electronic device 503 and transmitted to the computing device 508. Alternatively, the images may be captured directly at the computing device 508, for example, at the ICU 514.

At 606, the captured image is processed to display or otherwise provide diagnostic results of the processed fluid sample. In some embodiments, the processing step may be performed at a backend server such as the computing device 508. In others, the processing may happen client side, for example, at the mobile electronic device 503. Initially, the captured image(s) may be analyzed to detect panels. In instances where the system seeks to determine multiple analytes, a machine-learning model may be utilized. Referring to FIG. 13, the object-detection unit (ODU) 516 may apply one or more machine-learning models to one or more of the captured images to automatically determine the location on a given image where certain objects are present, such as panels. A machine-learning model may also classify any identified objects, such as classifying the object as a panel.

As described herein, the mobile electronic device 503 and computing device 508 may be a unitary device or may comprise a plurality of computing devices in communication. Therefore, the ODU 516 and/or database 518 may be within the mobile electronic device. Alternatively, the MED 503 may be separate from the computing device 508. The computing device may comprise the database 518 and/or ODU 516. The computing device may similarly comprise one or more computing devices. For example, the computing device 508 may comprise one or more computing device, including without limitation, cloud computing devices coupled together through communications network(s) 510. Therefore, once images are captured, the images may be uploaded to a cloud service where the images may be stored and processed according to embodiments described herein. Alternatively, the images may be stored and processed locally, such as at the MED 503.

As described herein the object-detection unit (ODU) 516 may apply one or more algorithms to one or more of the captured images to automatically determine the location on a given image where certain objects are present, such as panels, and in which to analyze for color variations. The object detection algorithms described herein are unique as embodiments described herein do not require specific shape matching. For example, the algorithms may not be pre-programmed to search for a specific shape, such as the described rectangular or square clover leaf described above with respect to the microfluidic device. Therefore, exemplary embodiments described herein may be used with one or more different microfluidic devices including those that are not yet introduced at the time of programming the ODU. The ODU therefore reduces outside restrictions on the form of the devices that may be analyzed according to embodiments described herein. Conventional systems may detect a specific shape or orientation and match to a pre-programmed or an expected shape to determine an area of interest for performing color processing. Such conventional systems impose restrictions on the use or modification of devices used with the conventional processing systems.

In various embodiments, a machine-learning model may be associated with one or more classifiers, which may be used to classify one or more objects. A classifier refers to an automated process by which an artificial intelligence system may assign a label or category to one or more data points. A classifier may include an algorithm that is trained via an automated process such as machine learning. A classifier typically starts with a set of labeled or unlabeled training data and applies one or more algorithms to detect one or more features and/or patterns within data that correspond to various labels or classes. The algorithms may include, without limitation, those as simple as decision trees, as complex as Naïve Bayes classification, and/or intermediate algorithms such as k-nearest neighbor. Classifiers may include artificial neural networks (ANNs), support vector machine classifiers, and/or any of a host of different types of classifiers. Once trained, the classifier may then classify new data points using the knowledge base that it learned during training. The process of training a classifier can evolve over time, as classifiers may be periodically trained on updated data, and they may learn from being provided information about data that they may have mis-classified. A classifier will be implemented by a processor executing programming instructions, and it may operate on large data sets such as image data and/or other data.

In one embodiment, when detecting for a single analyte, the ODU 516 may employ object-detection to detect the panels of the microfluidic device and samples therein. Object-detection as described herein may be different from shape matching of conventional systems. Exemplary embodiments described herein may implement a morphological shape detector that can determine an area of interest of any shape or size. Exemplary embodiments may, therefore, be used with different microfluidic devices having different chamber positions and/or shapes. In general, exemplary embodiments of the object detection method may include filtering the image. Filtering the image may include removing external anomalies, remove irregularities of the image through shape smoothing, contrast enhancement, etc. Exemplary embodiments of the object detection method may include boundary recognition to determine a largest target area of interest. Such boundary recognition may find true boundaries of the device, and erode color parameters and background parameters. Once the largest contour boundary is determined, then specific boundary points may be identified. Boundary points may include vertices or other apex or point defining a contour of a shape of the object boundary. Morphological shape detection may be used to fit any polygon or geometric shape to the boundary points identified by the defined object boundary. The identified shapes may be cut, cropped, scaled, repositioned, or a combination thereof for image processing and color detection. The target area of interest may then be defined as an interior or central portion of the identified shape defined by the morphological shape detection.

In an illustrative example, the object-detection may be based on the OpenCV library. An exemplary specific embodiment of the shape detection algorithm with associated images is provided with respect to FIGS. 16-20. Object detection may first obtain and sort one or more input image files.

Next, for any one image from an input image file, the image may be rotated (if necessary) and cropped in order to focus the diagnostic chambers (e.g., a cloverleaf pattern of diagnostic chambers) and respective diagnostic areas of the microfluidic device in order to produce a cropped image, as depicted in FIG. 16. The cropping may be performed according to a defined crop ratio. Exemplary embodiments may comprise the orientation and/or cropping as separate steps that may be performed in different orders. For example, a first cropping may be performed to create a working image in which the object detected simply takes up a desired area within the image, while orientation and/or additional cropping of the image may be performed after additional detection steps (such as the detection of the diagnostic chambers) as described herein.

Next, the diagnostic chambers may be detected. Exemplary embodiments may detect the diagnostic chambers by first filtering the image. Exemplary filtering may removing shadows from the cropped image. Shadows may be removed by using image dilation and absolute difference functionalities of OpenCV to obtain a normalized image as depicted in FIG. 17. Exemplary embodiments may dilute the image in order to remove color and/or background parameters from the image to permit true objects boundaries to be detected. Other image filtering may be used in combination with or in place of image dilation and absolute difference functions in order to filter the image. For example, shape smoothing may be used to remove irregularities from the object image.

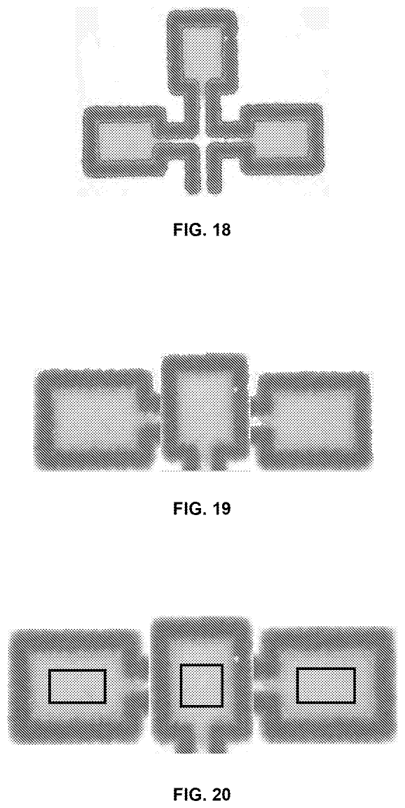

The normalized image may be further processed to determine the largest contour boundary. The system may therefore calculate a largest contour area-based contour. The largest contour boundary is determined based on an exterior boundary of the detected object and/or interior boundaries detected. The image may then be filtered to obtain an image of the isolated diagnostic chambers, as depicted in FIG. 18. Contours near the edges of the images may be ignored.

Exemplary embodiments may take the normalized image and largest contour boundary to determine boundary points. Exemplary boundary points may be determined based on contours of the detected boundary, such as in transitions or apex points of a boundary line. Morphological shape detection may be used based on the boundary points to fit a polygon or geometric boundary to the detected points. The shapes may thereafter be extracted for analysis. The extraction may include additional cropping, scaling, or repositioning of the images for analysis. In a specific example of an exemplary embodiment for determining boundary points to extract target areas of interest for analysis, the contoured image may run a shadow removal function for a second time, followed by extraction of each of the left, top, and right panel contour points from the image. The positioning of each panel may be determined by a target contour bounding box calculation to determine each panel's coordinates. The extracted panels, shown in FIG. 19, are then saved to a separate image file that may be further processed for detection or extraction of a color sample.

For detection, newly created extracted panel images are first sorted before further processing. To begin sample detection, the panels of a newly created extracted panel image are read. A central rectangular sample region bounding rectangle point is calculated by cropping image points according to a specified vertical and horizontal margin ratio, as seen in FIG. 20. The resultant cropped rectangle is then saved to a separate image file. A rectangle shape is used as an exemplary embodiment, however, the invention is not so limited. Any geometric target shape may be used, including a smaller ratio shape from the morphological shape detector fitting a polygon or other shape to the bounding points. Exemplary shapes may include squares, circles, ovals, rectangles, or other shape.

For extraction, the sample panels are processed similar to the method described above for detection. An extract color card sample function is performed, and the resultant sample is saved as a NumPy array.

Once the panels have been identified, a threshold and anchors of the image are determined.

The anchors of the image are reference points that are known to the system (corners, the colored dots etc.) that enable the system to anchor its surroundings and properly segment objects from the captured images. The threshold is the bounding rectangle point described above, cropped from the center of the reaction region of the diagnostic chambers of a microfluidic device.

Based on the determined threshold and anchors, the bounding boxes of the image are generated that isolate the region of interest in the image. In an expected scenario using the cloverleaf microfluidic device embodiment, three rectangles should be identified during the bounding process, one rectangle corresponding to each panel of a given microfluidic device. The remaining processed image is saved, temporarily, to memory at the mobile electronic device 503 for future access and retrieval. In another embodiment, the processed image may be temporarily saved to memory at the computing device 508. Once saved, the processed image may be uploaded to a cloud service for further processing. In yet another embodiment, only one panel may be identified and solely used during color processing.

Once an image has been processed to identify the region of interest, the processed image(s) is used in color processing to determine diagnostic results. The color processing may employ machine learning using color spectrum values of panels' Region of Interest (ROI) median pixel value as training data. For example, for an RGB spectrum, the ROI's RGB array would be taken, then the median taken from that (this applies to all different types of models for all the different sets of color spectrum channel combinations). The system is aiming to determine the 2^{nd} tier of most dominate colors (i.e., avoid the blue that the device is mainly comprised of). A given identified color may correspond to one or two types of results, depending on the type of diagnostic test with which the color is associated. For example, for metabolic tests, the colors are quantitative—a certain collection of RGB values represents a single quantitative number. Alternatively, for a binary diagnostic test (e.g., a positive or negative result) the presence or absence of a color (or colors) can indicate a positive or negative result.

The system determines the color in each rectangle by clustering the pixels and making a histogram, and then normalizing the histogram. After extracting the color as a color spectrum array, the system takes a median value of that array for the selected channel for which the test is seeking prediction. For example, if a particular test uses R channel as input, then the system will take a median value of R. If a test takes RGB channel as input, then a median RGB value is calculated. The system then determines the selected median value of the selected channel, then modulates the selected channel to a name. In some embodiments, the resulting image is stored at the mobile electronic device 503. In others, it may be transferred to and stored at the computing device 508.

The ODU 516 may be utilized to make the prediction described in the previous paragraph. Several models may be employed depending on the test performed. The algorithms used may include generalized linear models using polynomial transforms. The model algorithms may include linear regression, ridge regression, lasso regression, and ElastsicNet. Depending on the test, the extracted median color spectrum channel values may be supplied to any of the listed models. The prediction result, a concentration value, may be represented as a floating point number.

Any results of the image processing may be displayed in a graphical user-interface generated at the mobile electronic device 503 in some embodiments, or at the computing device 508 in others. In embodiments where the processing occurs at the computing device 508, the computing device may transmit at least a portion of the results to the mobile electronic device 503 for display. Such graphical-user interfaces may include various buttons, fields, forms, components, data streams, and/or the like, any of which may be used to visualize the results.

FIG. 14 illustrates an example of a suitable computing and networking environment 700 that may be used to implement various aspects of the present disclosure, such as the computing device 508. As illustrated, the computing and networking environment 700 includes a computing device, although it is contemplated that the networking environment of the computing and networking environment 700 may include one or more other computing systems, such as personal computers, server computers, hand-held or laptop devices, tablet devices, multiprocessor systems, microprocessor-based systems, set top boxes, programmable consumer electronic devices, network PCs, minicomputers, mainframe computers, digital signal processors, state machines, logic circuitries, distributed computing environments that include any of the above computing systems or devices, and the like.

Components of the computing and networking environment 700 (e.g., computer) may include various hardware components, such as a processing unit 702, a data storage 704 (e.g., a system memory), and a system bus 706 that couples various system components of the computer 700 to the processing unit 702. The system bus 706 may be any of several types of bus structures including a memory bus or memory controller, a peripheral bus, and a local bus using any of a variety of bus architectures. For example, such architectures may include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

The computer 700 may further include a variety of computer-readable media 708 that includes removable/non-removable media and volatile/nonvolatile media, but excludes transitory propagated signals. Computer-readable media 708 may also include computer storage media and communication media. Computer storage media includes removable/non-removable media and volatile/nonvolatile media implemented in any method or technology for storage of information, such as computer-readable instructions, data structures, program modules or other data, such as RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium that may be used to store the desired information/data and which may be accessed by the computer 700. Communication media includes computer-readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. For example, communication media may include wired media such as a wired network or direct-wired connection and wireless media such as acoustic, radio frequency (RF), infrared, and/or other wireless media, or some combination thereof. Computer-readable media may be embodied as a computer program product, such as software stored on computer storage media.

The data storage 704 includes computer storage media in the form of volatile/nonvolatile memory such as read only memory (ROM) and random access memory (RAM). A basic input/output system (BIOS), containing the basic routines that help to transfer information between elements within the computer 700 (e.g., during start-up) is typically stored in ROM. RAM typically contains data and/or program modules that are immediately accessible to and/or presently being operated on by processing unit 702. For example, in one embodiment, data storage 704 holds an operating system, application programs, and other program modules and program data.

Data storage 704 may also include other removable/non-removable, volatile/nonvolatile computer storage media. For example, data storage 704 may be: a hard disk drive that reads from or writes to non-removable, nonvolatile magnetic media; a magnetic disk drive that reads from or writes to a removable, nonvolatile magnetic disk; and/or an optical disk drive that reads from or writes to a removable, nonvolatile optical disk such as a CD-ROM or other optical media. Other removable/non-removable, volatile/nonvolatile computer storage media may include magnetic tape cassettes, flash memory cards, digital versatile disks, digital video tape, solid state RAM, solid state ROM, and the like. The drives and their associated computer storage media, described above and illustrated in FIG. 14, provide storage of computer-readable instructions, data structures, program modules and other data for the computer 700.

A user may enter commands and information through a user interface 710 or other input devices such as a tablet, electronic digitizer, a microphone, keyboard, and/or pointing device, commonly referred to as mouse, trackball, or touch pad. Other input devices may include a joystick, game pad, satellite dish, scanner, or the like. Additionally, voice inputs, gesture inputs (e.g., via hands or fingers), or other natural user interfaces may also be used with the appropriate input devices, such as a microphone, camera, tablet, touch pad, glove, or other sensor. These and other input devices are often connected to the processing unit 702 through a user interface 710 that is coupled to the system bus 706, but may be connected by other interface and bus structures, such as a parallel port, game port or a universal serial bus (USB). A monitor 712 or other type of display device is also connected to the system bus 706 via an interface, such as a video interface. The monitor 712 may also be integrated with a touch-screen panel or the like.

The computer 700 may operate in a networked or cloud-computing environment using logical connections of a network interface or adapter 714 to one or more remote devices, such as a remote computer. The remote computer may be a personal computer, a server, a router, a network PC, a peer device or other common network node, and typically includes many or all of the elements described above relative to the computer 700. The logical connections depicted in FIGS. 13-14 may include one or more local area networks (LAN), one or more wide area networks (WAN) and/or other networks, and combinations thereof. Such networking environments are commonplace in offices, enterprise-wide computer networks, intranets and the Internet.

When used in a networked or cloud-computing environment, the computer 700 may be connected to a public and/or private network through the network interface or adapter 714. In such embodiments, a modem or other means for establishing communications over the network is connected to the system bus 706 via the network interface or adapter 714 or other appropriate mechanism. A wireless networking component including an interface and antenna may be coupled through a suitable device such as an access point or peer computer to a network. In a networked environment, program modules depicted relative to the computer 700, or portions thereof, may be stored in the remote memory storage device.

Methods of Use

In some embodiments, the disclosed microfluidic device 1 is useful for detecting and quantifying target analytes and biomarkers present in a fluid sample, such as a biological or non-biological fluid sample. Suitable biological samples include but are not limited to blood, tissue, urine, sputum, vaginal secretions, anal secretions, oral secretions, penile secretions, saliva, and other bodily fluids. In other embodiments, the fluid sample may be a non-biological fluid, and the disclosed microfluidic device is useful for detecting and quantifying target analytes (e.g., chemical or biological contaminants) present therein. The fluid sample may be processed or unprocessed. Processing can include filtration, centrifugation, pre-treatment by reagents, etc. For example, a biological blood sample may be filtered to remove a component of the sample (e.g., whole blood may be filtered to remove red blood cells). A biological sample (e.g., tissue cells) or non-biological sample (e.g., soil) may also be mixed with a solution (e.g., distilled water or buffer) to form a fluid prior to depositing the sample onto the microfluidic device.

Non-limiting examples of target analytes that may be detected using the disclosed technology include antibodies, proteins (e.g., glycoprotein, lipoprotein, recombinant protein, etc.), polynucleotides (e.g., DNA, RNA, oligonucleotides, aptamers, DNAzymes, etc.), lipids, polysaccharides, hormones, prohormones, narcotics, small molecule pharmaceuticals, pathogens (e.g., bacteria, viruses, fungi, protozoa). In some embodiments, the target analyte includes one or more of: aspartate transaminase (AST), alkaline phosphatase (ALP), alanine aminotransferase (ALT), bilirubin, albumin, total serum protein, glucose, cholesterol, creatine, sodium, calcium, gamma glutamyl transferase (GGT), direct bilirubin, indirect bilirubin, unconjugated bilirubin, and lactate dehydrogenase (LDH). In some embodiments, the target analyte includes one or more components of a basic metabolic panel indicative of the medical status of the patient—e.g., glucose, blood urea nitrogen, calcium, bicarbonate, chloride, creatinine, potassium, and sodium. In some embodiments, the target analyte may be a chemical or biological contaminant, such as nitrogen, bleach, salts, pesticides, metals, toxins produced by bacteria, etc.

To use the disclosed microfluidic device 1, a fluid sample is deposited onto the microfluidic device. In some embodiments, the fluid sample is deposited at the common channel entry 7. In other embodiments, the fluid sample is deposited at a midway position of a fluid transfer channel 10—e.g., covering a location approximately half the length of the fluid transfer channel 10 between its initial end 8 and terminal end 9. In other embodiments, the sample is deposited directly onto the diagnostic paper, such as onto an upper surface of the paper or, if a through hole of a base is present, onto a lower surface of the paper. In some embodiments, the sample is deposited onto the center of the diagnostic paper, from which point the sample travels through the paper horizontally (e.g., when used with a base) or vertically (e.g., when used without a base) by capillary action. In some embodiments, the volume of the fluid sample is at least 5 μL or at least 10 μL, but no more than 60 μL, no more than 50 μL, no more than 40 μL, no more than 30 μL, or no more than 20 μL.

In some embodiments, the base 2 includes a filter (not shown) useful for filtering the sample before it reaches the diagnostic paper 13. The filter may be positioned at or adjacent the common channel entry 7 or at a midway position within a fluid transfer channel 10. In some embodiments, if the filter is positioned within a fluid transfer channel 10, it is spaced apart from (i.e., not in direct contact with) the diagnostic paper. Such positioning is intended to prevent the filter from drawing out the diagnostic components from the diagnostic paper, which could cause an assay reaction to undesirably occur on the filter paper rather than on the diagnostic paper alone. The filtered portion of the sample may flow from the filter through the fluid transfer channel 10 and into the corresponding diagnostic chamber 3. For example, a biological sample of whole blood may be deposited onto a filter to remove red blood cells, thereby allowing filtered serum to flow to the diagnostic paper.

A biological fluid sample may be deposited directly from a patient onto the device. For instance, a finger prick may be performed to produce a blood sample at the finger of a patient, which is then touched directly to the microfluidic device to deposit the sample at one of a variety of locations as described above. Alternatively, a biological or non-biological fluid sample may be deposited by an instrument, such as a pipette, capillary tube, eye dropper, or the like.

Once the fluid sample has been deposited on the microfluidic device 1 and has flowed (e.g., by capillary action) to the diagnostic paper 13 in a diagnostic chamber 3, a diagnostic assay may occur on the paper in the chamber. Non-limiting examples of suitable diagnostic assays include one or more of the following reactions: redox reactions, isothermal amplification, molecular diagnostics, immunoassays (e.g., ELISA), and colorimetric assays. In some embodiments, a diagnostic chamber may remain inactive so that no reaction occurs with the sample—e.g., as a control. The diagnostic assays can provide information for determining the presence and quantity of a variety of target analytes. For instance, diagnostic assays performed on a biological fluid sample may provide information indicative of corresponding conditions such as, but not limited to, liver function, metabolic function, infectious diseases, cell counts, bacterial counts, viral counts, and cancers. By providing a plurality of diagnostic assays in a single device, one fluid sample can be simultaneously subjected to a plurality of independent assay reactions that provide an informative landscape of data directed to multiple conditions of interest. In some embodiments, all of the diagnostic assays may be directed to a single condition of interest (e.g., liver disease, diabetes, contaminant levels etc.). In other embodiments, the diagnostic assays may be selected to provide a multifaceted profile of a patient (e.g., glucose levels, electrolyte levels, kidney function, etc.) or the tested fluid itself (e.g., contamination levels in a soil solution).

During a diagnostic assay, certain diagnostic component(s) will selectively associate with a corresponding target analyte. As used herein, “selectively associates” refers to a binding reaction that is determinative for a target analyte in a heterogeneous population of other similar compounds. For example, the diagnostic component may be an antibody or antibody fragment that specifically binds to a target antigen. Non-limiting examples of suitable diagnostic components include 5-bromo-4-chloro-3-indolyl phosphate (BCIP), alpha-ketoglutarate, glucose oxidase, horseradish peroxidase, cholesterol oxidase, hydroperoxide, diisopropylbenzene dihydroperoxide, an apolipoprotein B species, 8-quinolinol, or monoethanolamine, 2,4-suraniline, 2,6-dichlorobenzene-diazonium-tetrafluoroborate, bis (3′,3″-diiodo-4′,4″-dihydroxy-5′,5″-dinitrophenyl)-3,4,5,6-tetrabromosulfonephtalein (DIDNTB), a phenolphthalein anionic dye, nitro blue tetrazolium (NBT), methyl green, rhodamine B, 3,3′,5,5′-tetramethylbenzidine, a diaphorase, methylthymol blue, a diazonium salt, and oxalacetic acid.

In some embodiments, the diagnostic component(s) include a visual indicator that exhibits a colorimetric and/or fluorometric response in the presence of a target analyte. For example, such visual indicators may become colored in the presence of the analyte, change color in the presence of the analyte, or emit fluorescence, phosphorescence, or luminescence in the presence of the analyte, or a combination thereof.

In some embodiments, it takes about 1 minute or less (e.g., less than 70 seconds, less than 60 seconds, less than 50 seconds, less than 40 seconds, less than 30 seconds, less than 20 seconds, or less than 10 seconds) for a biological sample to reach each diagnostic chamber after being deposited onto a recessed microfluidic device disclosed herein. In some embodiments, it takes about 2 minutes or less (e.g., less than 145 seconds, less than 120 seconds, less than 100 seconds, less than 80 seconds, less than 60 seconds, less than 40 seconds, or less than 30 seconds) for a biological sample to adequately fill each diagnostic chamber after being deposited onto the microfluidic device. In some embodiments, a recessed microfluidic device (e.g., with 3-6 diagnostic chambers) is configured such that about 90 μL or less (e.g., less than 90 μL, less than 80 μL, less than 70 μL, less than 60 μL, less than 50 μL, less than 40 μL, less than 30 μL, or less than 20 μL) of a whole blood sample will reach each diagnostic chamber within about 60 seconds, within about 45 seconds, within about 30 seconds, within about 20 seconds, within about 15 seconds, within about 10 seconds, or within about 7 seconds. In some embodiments, a recessed microfluidic device (e.g., with 3-6 diagnostic chambers) is configured such that about 90 μL or less (e.g., less than 90 μL, less than 80 μL, less than 70 μL, less than 60 μL, less than 50 μL, less than 40 μL, less than 30 μL, or less than 20 μL) of a whole blood sample will adequately fill each diagnostic chamber within about 120 seconds, within about 90 seconds, or within about 60 seconds.

In some embodiments, the assay reactions conducted on any embodiment of a microfluidic device disclosed herein will be completed within about 60 minutes or less from the time the fluid sample is deposited onto the microfluidic device—e.g., about 60 minutes or less, about 50 minutes or less, about 40 minutes or less, about 30 minutes or less, or about 20 minutes or less. The time of fluid sample deposition can be measured from the time the sample contacts the diagnostic paper or from the time the sample contacts a filter paper spaced apart from or stacked on top of the diagnostic paper. After completion of the assay reaction(s), the microfluidic device can be easily and rapidly imaged, thus providing full diagnostic results extremely quickly.

For example, an image of the reacted microfluidic device may be captured and/or analyzed according to applications described above. More specifically, the reacted microfluidic device may be placed on the position indicator 26 of the bottom panel 21 of a viewing box 30. The viewing box may then be closed. With the one or more internal light source(s) illuminated, the camera of a mobile electronic device can be positioned over the uppermost top panel viewing aperture such that the reacted microfluidic device can be visually observed by the user via the camera. An image of the reacted microfluidic device may then be captured and processed according to embodiments described herein to general diagnostic results corresponding to the sample deposited and reacted on the microfluidic device. Those results may be electronically and securely stored within the application with respect to the fluid sample source and its identifying information.

### EXAMPLES

The present invention is next described by means of the following examples. The use of these and other examples anywhere in the specification is illustrative only, and in no way limits the scope and meaning of the invention or of any exemplified form. Likewise, the invention is not limited to any particular preferred embodiments described herein. Indeed, modifications and variations of the invention may be apparent to those skilled in the art upon reading this specification, and can be made without departing from its spirit and scope. The invention is therefore to be limited only by the terms of the claims, along with the full scope of equivalents to which the claims are entitled.

### Example 1: Microfluidic Device Scanning Process for Patient Diagnostic Use

This example describes a process for using a microfluidic device and viewing box assembly of the present disclosure. In this example, the mobile electronic device is a smartphone having a microfluidic device scanning application installed; and the fluid sample is a biological fluid sample obtained from a patient and deposited onto the microfluidic device.
(1) Login: Open the microfluidic device scanning application on the smartphone to display a login page. On the login page, the user enters patient identifying information, such as an e-mail and password. After that information is entered, the application proceeds to a Create screen, where the user can select an option to register a new patient. The user may be the patient, a technician, a medical services provider, or other individual.
(2) Register the Patient: On a subsequent Registration screen, the user enters additional patient identifying information, such as first name, last name, medical records number, date of birth, other notes, phone number, and a number corresponding to the microfluidic device to be used.
(3) Link Microfluidic Device QR Code with Registered Patient: On a subsequent Create Patient screen, the user selects an option to “Add QR code and start time.” The camera on the smartphone is then used to capture an image of the QR code on the microfluidic device. The application reads the QR code, and thereby links the patient identifying information to the information specific to the microfluidic device, such as the relevant assay to be conducted. A message is displayed to the user confirming that the device QR code was successfully linked with the patient identifying information. Once the QR code is linked, the test will start with a timer that is set for a specified duration—here, 50 minutes. When the timer starts, biological sample is deposited onto the microfluidic device.
(4) Image Capture: After the timer for the assay reaction expires, the reacted microfluidic device is positioned over the position indicator on the bottom panel of the viewing box. At this point, the microfluidic device is ready to be imaged. Using the microfluidic device scanning application, the camera of the smartphone activates with a highlighted yellow box displayed in the image preview. A user positions the camera such that the microfluidic device can be seen within the displayed highlighted box. The smartphone is then used to capture the image, which is processed by the application to generate quantified results. A message is displayed to the user confirming that the image processing was successful and that the diagnostic results are available.
(5) Explore Results: The diagnostic results of the assay are then displayed on a Results screen, which may include information such as the patient's name, target analyte concentration (here, bilirubin (BIL): 0.38742 mg/dl), and the time and date of the assay (here, Dec. 17, 2020—1:08 pm). The results are stored on the smartphone and/or uploaded to a server. The user may scan the QR code to view the patient results, as needed.

### Example 2: Microfluidic Device and Smartphone Technology for Colorimetric Quantification of Bilirubin in Serum

This example relates to the use of a paper-plastic hybrid microfluidic device of the present disclosure to quantify total bilirubin in human serum using image processing and machine learning technology. Total bilirubin values have been used as a potential marker to pre-screen and diagnose various liver-based diseases such as jaundice, bile obstruction, liver cancer etc. The biochemical assays are deposited in absorbent paper pads that act as reaction zones when serum is added. A dedicated app in smartphones captures images of the colorimetric changes on the pad and converts them into quantitative values of the bilirubin. The range of bilirubin concentration that can be quantified using the device is from 0.3 mg/dL to 7.0 mg/dL. The precision, limit of detection, linearity, stability, and comparison with a predicate are studied in this example in accordance with clinical and laboratory standards institute (CLSI) protocols. The results confirm that the microfluidic device can be used as an inexpensive alternative to conventional bilirubin testing. With its level of precision, ease-of-use, long shelf-life, and the short turnaround time, it provides significant value in point-of-care and clinical settings.

In this example, the microfluidic device detects bilirubin using the Diazo-dye method stabilized on a paper-based device. Total bilirubin found in the serum reacts with the diazo reagent in the presence of accelerators to form azobilirubin under acidic conditions. The intensity of the color is quantified using an iOS application in conjunction with an iPhone camera, and an engineered viewing box to standardize lighting.

Device and box manufacturing: The device includes an acrylic backbone manufactured using a laser cutting machine (BOSS LS1630). Alpha cotton linter cellulose is the material used for the absorbent paper-pads and they are securely embedded in the open acrylic panels, acting as the reaction zones. Accompanying the device is an engineered closed acrylic box, also manufactured using the laser cutting machine. The box acts as a barrier to keep out ambient light and minimizes external atmospheric interference. The box contains an inbuilt light source and a dedicated indent to keep the device in an optimal environment after the sample is added. The box contains a holder to place the phone with the camera for image processing.

Preparation of reagents: The assay described herein is a modified version of the Jendrassik-Grof Diaz® method. A solution of sulfanilic acid (Sigma #822338), sodium nitrite (Alfa Aesar #A18668) and accelerants caffeine (Sigma #27602), sodium acetate (Sigma #S2889), and sodium benzoate (Sigma #109169), are used to prepare the reagent. The sulfanilic acid, sodium benzoate and accelerants are mixed in a ratio of 4:1:4, respectively, and are deposited and dried on the paper pads on the device.

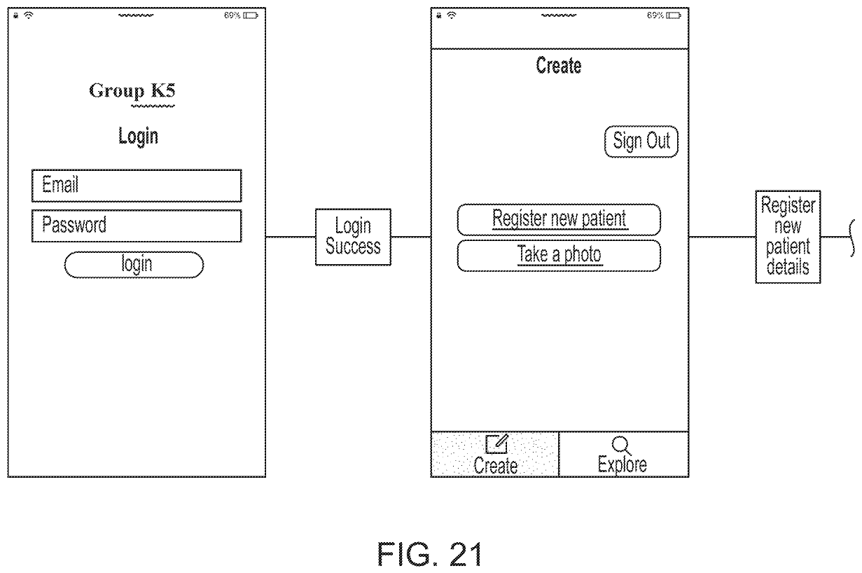

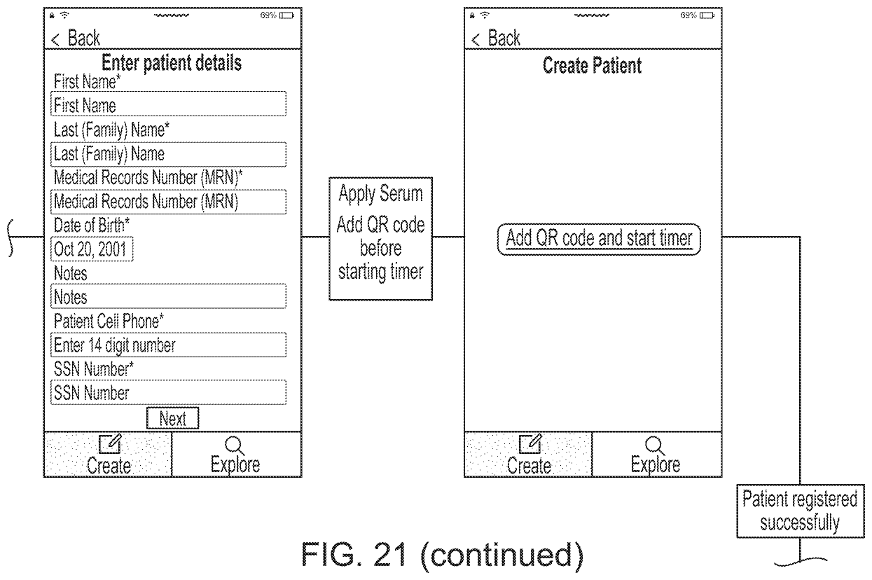

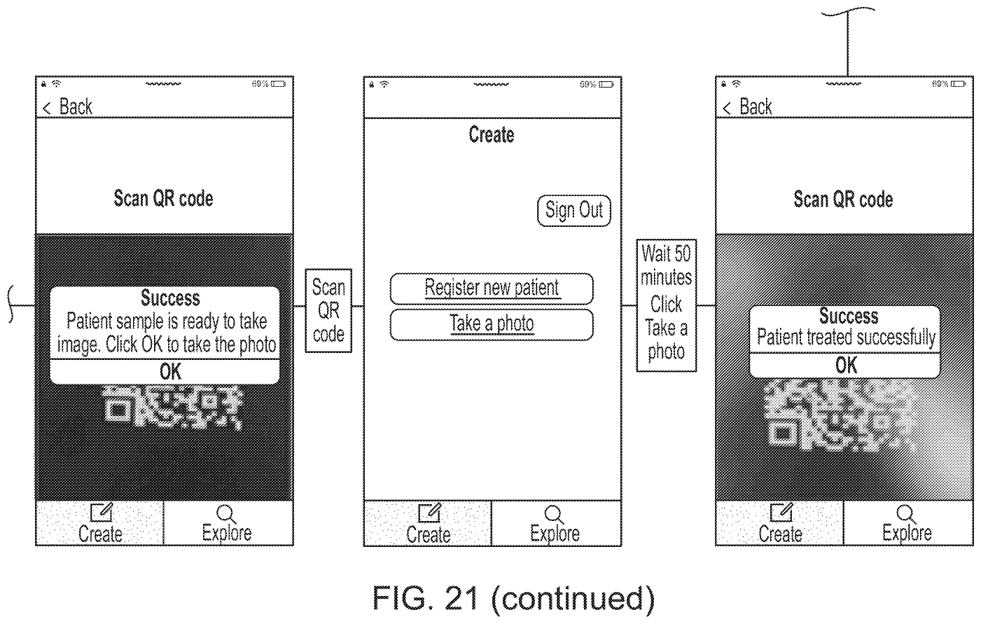

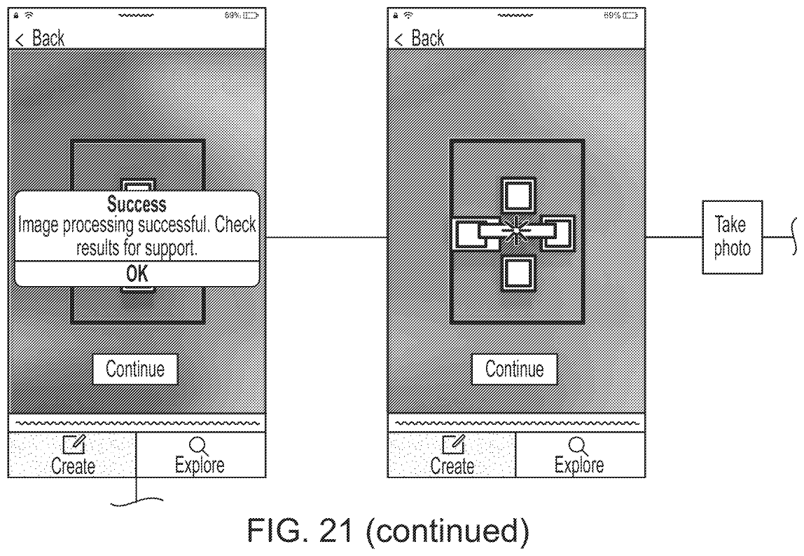

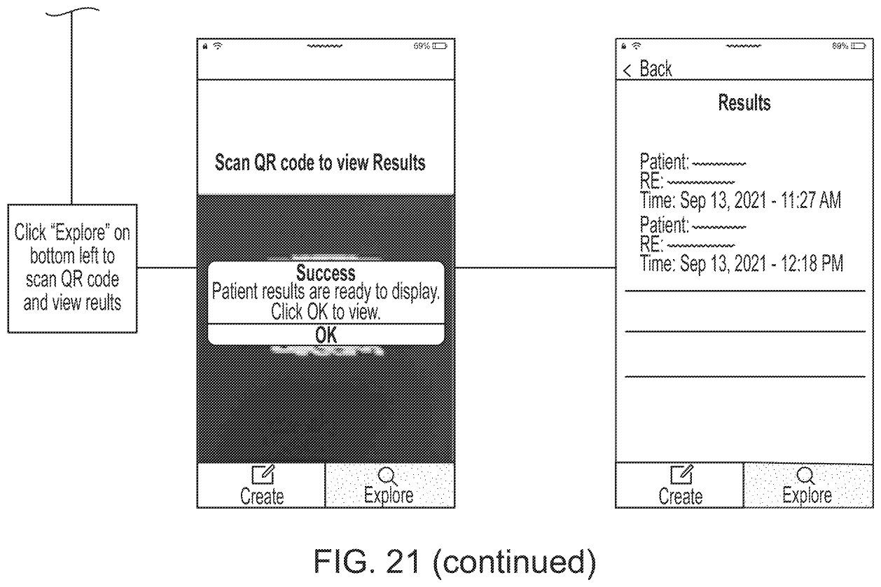

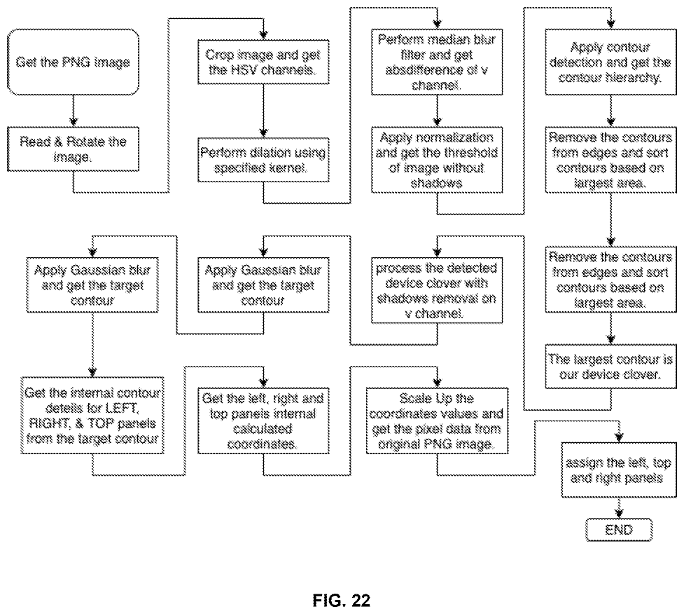

Procedure for assay, iOS app development and image processing: The patient's serum is pipetted onto the paper pads in the device and allowed to react with the reagents stabilized on the pad for 50 minutes. The color of the absorbent pad changes with increasing concentrations of bilirubin. After the reaction period, the device is placed in the indent inside the viewing box. The phone is placed on the specified location with the camera aligned with the pinhole on the top of the box. An image detection app is developed on the iOS 14.3 platform to capture and transfer the image as well as to show the results. For instance, the iOS application is used to scan a QR code on the device, filling in patient details, and the phone is used to capture the device image. The app runs using a cloud-based software in compliance with HIPAA protocols. This app acts as the user interface and is expected to function adequately on any equivalent device, including and after iPhone 7. A detailed application workflow is shown in FIG. 21. The image processing workflow to extract the region of interest (ROI) from the individual panels is shown in FIG. 22.

After capturing the image, the user data is uploaded to the AWS S3 data storage. After uploading the user data, the backend API makes the execution call to the python routine which further processes the respective uploaded image. The backend post-processes the captured DNG image file, converting it to a PNG image for further processing. After successful conversion of the DNG to PNG image form, the power of computer vision (specifically OpenCV toolkit) is used to further detect the region of interest in the given image. After getting the median color channel spectrum values, it is given as the input to the machine learning model for bilirubin. The model predicts the concentration of bilirubin using the color channel input. For the purpose of the following studies, only a single panel (always the same) of each device is used for testing in order to optimize image processing. An iPhone 7 was used as the baseline hardware.

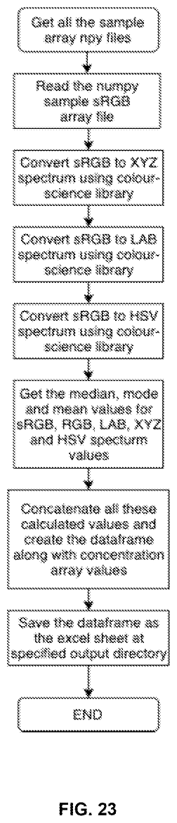

FIG. 23 shows the workflow for calculating the median of the color spectrum arrays obtained from the ROI that was used to develop machine learning models. The model is developed and selected based on the accuracy of prediction, with minimal error and robust r2 values. After obtaining the values, the median color channel spectrum values are run through the underlying machine learning model developed for bilirubin quantification to predict the concentration of the sample. Predicted output for different concentrations of bilirubin was chosen from the trained model. Once the prediction has been made, the output is transferred to a given node which connects with a database server and is able to display the value to the user on the app.

Precision studies: A reproducibility study was performed to determine multi-site precision of the microfluidic device. The protocol for the study was developed using the guidelines from CLSI document EP05-A3. Multiple lots of devices in multiple sites with different operators were used to perform this study. Serum samples with spiked bilirubin were used for this test. The same concentrations of bilirubin were used for the entire 5-day study at each of three different laboratory sites. The five bilirubin concentrations used were: 0.4 mg/dL, 0.2 mg/dL, 0.98 mg/dL, 1.51 mg/dL and 2.04 mg/dL (identified here as samples P1 through P5, respectively). The same samples were used in all three sites to carry out a 5-day study with 5 sample replicates each day. Devices from a single lot are used to perform the study at one site.

A repeatability study was performed to determine within-laboratory precision of the device. The protocol for the study was developed using the guideline from CLSI document EP05-A3. Multiple lot devices in a single site were used to perform this study. Serum samples (Lee Biosolutions) with spiked bilirubin concentration were used in this study. In total, 10 different samples were used in this study, where two samples will be tested each day over a period of 20 days. The concentrations used for testing are: 0.2 mg/dL (Sample A); 0.4 mg/dL (Sample B); 0.97 mg/dL (Sample C); 1.50 mg/dL (Sample D); 2.03 mg/dL (Sample E); 3.33 mg/dL (Sample F); 4.07 mg/dL (Sample G); 5.64 mg/dL (Sample H); 6.50 mg/dL (Sample I); and 7.42 mg/dL (Sample J). The same sample was used for a 20-day experiment with 2 runs each day and 2 replicates. The 2 daily runs were performed in the morning and evening (or at least 4 hours apart). The same lot and same conditions were used while testing a given sample.

Limits of Detection: Limit of Blank (LoB) and Limit of Detection (LoD) protocols were prepared with reference to CLSI-EP17 A2. LoD is defined as the lowest concentration of the analyte that can be detected consistently. The blank sample has an analyte concentration lower than the LoD and is prepared by diluting the serum sample. The LoB is termed as the highest concentration that could be observed with a blank sample. Two reagent lots were used to do the LoB testing and the test was carried out over 3 days. Four blank samples were prepared to perform the experiment. The concentration of each of these samples was: 0.08 mg/dL (Blank 1), 0.11 mg/dL (Blank 2), 0.14 mg/dL (Blank 3), and 0.17 mg/dL (Blank 4). Each sample was tested for 2 replicates in each reagent lot.

For the lower LoD test, five samples were prepared with the suspected lowest level of detection. Each sample was tested for 5 replicates (5 devices) over 5 days. Two reagent lots were used for testing. 3 replicates were tested for Lot A, and 2 for Lot B. The concentrations of these samples were: 0.27 mg/dL (Sample 1), 0.3 mg/dL (Sample 2), 0.33 mg/dL (Sample 3), 0.35 mg/dL (Sample 4), and 0.37 mg/dL (Sample 5).

Linearity: The linearity test guideline describes the statistical process for determining the linearity of a quantitative measurement procedure. A primary objective is to determine the concentrations when a method becomes nonlinear and the extent of nonlinearity at that level. The linearity test protocol and guideline were prepared according to CLSI EPO6-A. The following steps were followed to perform linearity tests. Serum samples of various concentrations of bilirubin were used for this study. Seven samples were selected in which the concentrations of each sample were kept equidistant or such that there is an observable relationship between the samples. The concentrations tested were: 0.2 mg/dL, 1.0 mg/dL, 1.8 mg/dL, 2.6 mg/dL, 3.4 mg/dL, 4.2 mg/dL, and 5.0 mg/dL (Samples 1-7, respectively). The samples were tested in the microfluidic device. Each sample was tested in duplicates. The known concentration and its corresponding results were noted for the further analysis.

Predicate Device Comparison: This study compares the performance of the subject microfluidic device with a comparative measurement procedure (predicate device). The microfluidic device results were compared to Roche cobalt c311 results, termed as the predicate method. All clinical samples were collected from Access Biologics, CA. A total of 57 samples were tested to perform procedure comparison. The study was done over the span of a week. Multiple test lots were used to perform this protocol.

Shelf-life Study: A 6-month study of the shelf-life of the microfluidic device with the assay was tested using spiked serum samples, once every four weeks. The concentrations of the serum samples was 1.5 mg/dL, 4.0 mg/dL, and 7.0 mg/dL. Duplicates were tested for each concentration with each of the two lots of devices. Spiked serum samples above were prepared and stored as aliquots for each test in −20° C. and thawed at room temperature before each test.

Results and Discussion

Precision: All of Samples P1 to P5 showed minimal variance from site to site over the course of the study. Standard deviation (SD) and % Covariance (% CV) was calculated for repeatability, within-laboratory precision, and reproducibility (Table 1). These values give the device's precision profile. The repeatability (within-day precision) SD corresponds to Verror, within-laboratory precision corresponds to Verror and Vday, and the reproducibility (between-site precision) corresponds to all three: Verror, Vday, and Vsite.

TABLE 1

| Sample | Mean(mg/dL) | V error (within day) | V day (between-day) | V site (between-site) |
|---|---|---|---|---|
| P1 (0.4 mg/dL) | 0.392 | 0.012 | 0.001 | 0.001 |
| P2 (0.2 mg/dL) | 0.338 | 0.022 | 0.003 | 0.00 |
| P3 (0.98 mg/dL) | 1.178 | 0.100 | 0.004 | 0.001 |
| P4 (1.51 mg/dL) | 1.607 | 0.111 | 0.016 | 0.001 |
| P5 (2.04 mg/dL) | 2.313 | 0.247 | 0.021 | 0.002 |

Similarly, in the single-site study, 7 of the 10 tested samples showed very minor variation (Table 2).

TABLE 2

| Sample Description | Mean Value | V error | V run | V day |
|---|---|---|---|---|
| sample A (0.2 mg/dL) | 0.30 | 0.01 | 0.00 | 0.00 |
| sample B (0.4 mg/dL) | 0.49 | 0.02 | 0.00 | 0.01 |
| sample C (0.97 mg/dL) | 0.79 | 0.11 | 0.04 | 0.02 |
| sample D (1.50 mg/dL) | 1.61 | 0.41 | 0.41 | 0.04 |
| sample E (2.03 mg/dL) | 2.07 | 0.20 | 0.14 | 0.84 |
| sample F (3.33 mg/dL) | 3.93 | 1.40 | 1.15 | 0.17 |
| sample G (4.07 mg/dL) | 4.64 | 1.60 | 1.29 | 0.94 |
| sample H (5.64 mg/dL) | 6.42 | 1.91 | 1.02 | 0.34 |
| sample I (6.50 mg/dL) | 6.41 | 1.48 | 0.88 | 0.16 |
| sample J (7.42 mg/dL) | 7.34 | 0.47 | 0.45 | 0.06 |

Limits of Detection: The bilirubin LoB was calculated using non-parametric analysis. Rank position was calculated for each lot using the equation: Rank Position=0.5+B*Pct_{B}, wherein B is the number of blank measurements per reagent lot and Pct_{B} is the corresponding percentile (0.95 calculated using Type I error risk of α=0.05). Rank positions are integer values and interpolated as 21 and 22 for Lot 1 and 23, and 24 for Lot 2. The highest LoB (0.37 mg/dL) was used to calculate the LoD. For LoD, Lot 1 was calculated from 75 samples and Lot 2 was calculated from 50 samples. The LoD was calculated using the equation: LoD=cₚSD_{L}, wherein cₚ is the multiplier that gives the 95th percentile of the normal distribution and SD_{L} is the pooled SD per reagent lot. The LoD for bilirubin was 0.48 mg/dL. This result is consistent with the guidelines in CLSI document EP17 based on the proportions of false positives less than 5% and false negatives less than 5% with 150 low level samples and 48 blank samples, and LoB of 0.37 mg/dL.

Linearity: The concentrations for each sample and the differences between the replicates using the subject microfluidic device were measured. Linearity using regression analysis was determined. To prove linearity, as per CLSI guidelines, the non-linearity of the system was evaluated. The nonlinear coefficients were analyzed which are b2 in second order polynomial regression and b2 and b3 in third order polynomial regression. The second order did not have any nonlinear components and the system was linear. In third order fitting, one coefficient exceeds the criterion of 2.228 for 10 degrees of freedom. The second order model has much lower standard error than third order which proves that first order and second order prove better fitting than the third order. The predicted results for the first and second order polynomial regression show that the percentage difference was less than the laboratory criterion of 20% which provides evidence that the system is linear.

Comparison with predicate device: A comparison of the subject microfluidic device and the predicate device was conducted using a difference plot for each sample and analyzing the distribution of the difference. The difference value is calculated by taking the difference between predicate concentration value and subject microfluidic device predicted value for each sample. Since the difference has a skewed vertical distribution, bias estimation was computed from the median of difference values as 0.02 mg/dL for the measured range of 0.5-1.4 mg/dL. The 95% CI is calculated using the Wilcoxon Signed Rank Test as −0.05 to 0.05 mg/dL. The predefined bias criterion for equivalence was set to be ±0.1 mg/dL. The criterion for equivalence was met for the range of concentrations measured.

Shelf-life study: Results from the 6-month shelf-life study showed no significant (p<0.05) changes within the samples over the 24 weeks, except between Week 0 and Week 8 for 1.5 mg/dL and Week 12 and Week 24 for 4 mg/dL. However, slight changes in the predicted values could merely be due to the variation in serum sample aliquots used for the study.

Conclusion: The subject microfluidic device as disclosed herein provides an efficient and inexpensive paper-based device for colorimetric quantification of analytes, such as bilirubin, using a phone camera and backend image processing algorithms. It is easy to use, with a 50 min turnover time as demonstrated in the present example, making the device highly useful in POC settings. Additionally, the machine learning technology to quantify colorimetric biochemical processes is applicable to determine various biomarker levels for diagnostic and prognostic purposes.

### Example 3: Microfluidic Device without Base for Quantification of Aspartate Transaminase

This example relates to the use of an embodiment of the disclosed microfluidic device and image processing technology to reliably quantify aspartate transaminase (AST) in blood obtained from a fingerstick. AST is an enzyme that plays an important role in amino acid metabolism. Elevated levels of AST in the blood often help diagnose liver damage. The ratio of AST to other critical liver function markers such as Alanine Transaminase (ALT) and Bilirubin have potential for diagnosing several liver-disease pathologies. Hence, the disclosed technology can be used for the translational applications of several other disease biomarkers as well.

AST catalyzes the transfer of the α-amino group between aspartate and ketoglutarate: α-ketoglutarate+Aspartate→Oxalocetate+Glutamate.

The reaction between oxaloacetate and diazonium salt solution (Fast Violet B) provides for colorimetric quantification of AST (Babson et al., Clinica Chimica Acta 7, 2:199-205 (1962)). The image detection and model for colorimetric quantification can be modified for this AST-specific microfluidic device.

Methods: As shown in FIG. 24, the device was made using two 2 cm diameter layers: a top filter paper layer (made from D23, TC-1, MF1, or F5) for plasma separation from whole blood, and a bottom paper diagnostic layer (made from borosilicate glass microfiber material) for an AST catalyzing reaction. The top layer is a plasma separation membrane that traps red blood cells in whole blood, allowing plasma to run through. The bottom layer holds the reagents or diagnostic components of the colorimetric biochemical reaction. The two layers were stacked together and completely laminated (with 5 mm punch holes on both sides as inlet and outlet so that the sample could vertically flow directly into and through the paper layers). As shown in FIG. 25, the assembled device was prepared with diagnostic components deposited through the bottom aperture onto the diagnostic test layer and allowed to dry.

To quantify AST from a fingerstick of whole blood, a lancet is used to prick the finger of the subject. A thin heparinized capillary tube (max capacity 30 μL) is used to collect the blood and the 30 μL drop is placed through the top inlet aperture onto the filter paper layer. Subsequently, 30 μL of 1×PBS buffer is provided to chase the plasma onto the bottom layer. The AST in the plasma then catalyzes the enzymatic reaction with the reagents (diagnostic components) in the bottom layer to produce a product that can be colorimetrically quantified. The plasma separation and reaction was allowed to proceed at 37° C. for 20 mins before a dye (provided in an ampule) was added through the bottom outlet aperture and held at 37° C. for an additional 10 mins, allowing for the color change to occur. Once the color develops in the set time, the device is placed in a viewing box as disclosed above and a phone camera can be used to take an image of the bottom outlet of the device. The app and algorithm are then used to quantify the color change as a concentration of AST.

Four test samples were run corresponding to the four types of filter membrane, each alongside a control (no assay). The resultant colorimetric change may be quantified using the image processing technology disclosed herein.

Results and Discussion: Successful separation of plasma and colorimetric change was observed with all test samples, each of which showed significant color change compared to the controls without reagents. Quantification of AST in the blood can be determined using the image processing technology disclosed herein, including the quantification method described in Example 2.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements and methods which, although not explicitly shown or described herein, embody the principles of the disclosure and are thus within the spirit and scope of the present disclosure. From the above description and drawings, it will be understood by those of ordinary skill in the art that the particular embodiments shown and described are for purposes of illustrations only and are not intended to limit the scope of the present disclosure. References to details of particular embodiments are not intended to limit the scope of the disclosure.

All references cited and/or discussed in this specification are incorporated herein by reference in their entireties and to the same extent as if each reference was individually incorporated by reference.

1. A recessed microfluidic device, comprising:
a base having an upper surface and a lower surface, the upper surface comprising at least one recessed fluid transfer channel in fluid communication with a corresponding diagnostic chamber, wherein the diagnostic chamber comprises a recessed area substantially surrounded by a raised frame; and
diagnostic paper sized to fit within the recessed area of the diagnostic chamber, wherein the diagnostic paper includes one or more diagnostic components provided thereon.

2. The recessed microfluidic device of claim 1, comprising three recessed fluid transfer channels fluidically coupled to a common channel entry.

3. The recessed microfluidic device of claim 1, wherein the diagnostic paper is a single layer sheet of hydrophilic, porous paper.

4. The recessed microfluidic device of claim 1, wherein the diagnostic paper is filter paper or chromatography paper.

5. The recessed microfluidic device of claim 1, wherein the one or more diagnostic components are selected from reagents, dyes, probes, stabilizers, catalysts, anti-coagulants, lysing agents, nanoparticles, diluents, and combinations thereof.

6. The recessed microfluidic device of claim 1, wherein at least one diagnostic component is capable of selectively associating with an analyte selected from aspartate transaminase, alkaline phosphatase, alanine aminotransferase, bilirubin, albumin, total serum protein, glucose, cholesterol, creatine, sodium, calcium, gamma glutamyl transferase, direct bilirubin, indirect bilirubin, unconjugated bilirubin, and lactate dehydrogenase, glucose, blood urea nitrogen, calcium, bicarbonate, chloride, creatinine, potassium, and sodium.

7. The recessed microfluidic device of claim 1, further comprising a filter in fluid communication with at least one fluid transfer channel, wherein the filter is spaced apart from the diagnostic paper.

8. The recessed microfluidic device of claim 1, wherein the base further comprises an extension having an upper surface on which an identifying indicator is provided.

9. The recessed microfluidic device of claim 8, wherein the identifying indicator comprises a QR code or barcode.

10. A microfluidic device and viewing box assembly, comprising:
a microfluidic device comprising a single layer of diagnostic paper that includes one or more diagnostic components provided thereon; and
a viewing box comprising a bottom panel, one or more top panels, four or more side panels, and one or more internal light source(s), wherein each top panel comprises a viewing aperture;
wherein the microfluidic device is configured to fit within the viewing box when the viewing box is assembled.

11. The assembly of claim 10, wherein the top panel viewing apertures are the only openings through which light may enter the viewing box.

12. The assembly of claim 10, wherein at least the interior of the viewing box is made entirely from solid, opaque material.

13. The viewing box assembly of claim 10, wherein an interior surface of the bottom panel includes a position indicator marking to identify a desired placement position of the microfluidic device.

14. The viewing box assembly of claim 10, wherein the microfluidic device is a recessed microfluidic device, comprising a base having an upper surface and a lower surface, the upper surface comprising at least one recessed fluid transfer channel in fluid communication with a corresponding diagnostic chamber, wherein the diagnostic chamber comprises a recessed area substantially surrounded by a raised frame; and the diagnostic paper is sized to fit within the recessed area of the diagnostic chamber.

15. The viewing box assembly of claim 10, wherein the microfluidic device comprises: a top layer of diagnostic paper and a bottom layer of filter paper; and lamination layers provided on a top surface of the top layer and a bottom surface of the bottom layer, wherein the lamination layers are adhered together and the lamination layers comprise aligned apertures configured to permit vertical flow of a fluid sample deposited through the top aperture.

16. A method of detecting and quantifying a target analyte in a fluid sample, comprising the steps of:
(a) obtaining a fluid sample;
(b) depositing the fluid sample onto a microfluidic device comprising a single layer of diagnostic paper that includes one or more diagnostic components provided thereon;
(c) waiting for a predetermined period of time during which the fluid sample flows to each diagnostic chamber where a reaction occurs between the target analyte in the sample and the one or more diagnostic components;
(d) placing the reacted microfluidic device into a viewing box comprising a bottom panel, one or more top panels, four or more side panels, and one or more internal light source(s), wherein each top panel comprises a viewing aperture, and the viewing apertures are the only openings through which light may enter the viewing box;
(e) placing a camera of a mobile electronic device over the viewing aperture and capturing an image of the reacted microfluidic device while illuminated by the one or more internal light source(s);
(f) transmitting, by the first electronic device, the image to a second electronic device via a communication network;
(g) applying, by the second electronic device, one or more object detection models to the image to generate one or more diagnostic results pertaining to the fluid sample;
(h) transmitting, by the second device, at least a portion of the diagnostic results to the first electronic device; and
(i) displaying, by the first electronic device, a visual representation corresponding to the at least a portion of the diagnostic results on a display of the first electronic device.

17. The method of claim 16, wherein the fluid sample is a biological fluid sample.

18. The method of claim 16 or 17, wherein the predetermined period of time is about 60 minutes or less from the time the fluid sample is deposited onto the microfluidic device.

19. The method of claim 16, wherein the results of step (g) comprise diagnostic results.

20. The method of claim 16, wherein the microfluidic device includes an extension, and the method further comprises applying an identifying indicator onto an upper surface of the extension.

21. The method of claim 16, wherein the processing of the image comprises clustering pixels of the image into a histogram sorted according to color values.

22. The method of claim 16, wherein sorting into a histogram according to color values comprises: determining an RGB value, modulating the RGB value to a HEX value, and modulating the RGB value to a corresponding color name.

23. The method of claim 16, wherein the captured image is received from a mobile device and a graphical user interface (GUI) is displayed at the mobile electronic device.

24. The method of claim 16, wherein the microfluidic device is a recessed microfluidic device, comprising a base having an upper surface and a lower surface, the upper surface comprising at least one recessed fluid transfer channel in fluid communication with a corresponding diagnostic chamber, wherein the diagnostic chamber comprises a recessed area substantially surrounded by a raised frame; and the diagnostic paper is sized to fit within the recessed area of the diagnostic chamber.

25. The method of claim 16, wherein the microfluidic device comprises: a top layer of diagnostic paper and a bottom layer of filter paper; and lamination layers provided on a top surface of the top layer and a bottom surface of the bottom layer, wherein the lamination layers are adhered together and the lamination layers comprise aligned apertures configured to permit vertical flow of a fluid sample deposited through the top aperture.
